(19) United States
(12) Patent Application Publication
(10) Pub. No.: US 2008/0125469 A1
(43) Pub. Date: May 29, 2008

(54) Thiadiazole compounds and methods of use thereof

(75) Inventors: Matthew G. Bursavich, Tuckahoe, NY (US); Sabrina Lombardi, New Haven, CT (US); Adam M. Gilbert, Congers, NY (US)

Correspondence Address: WilmerHale/Wyeth, 60 STATE STREET, BOSTON, MA, 02109, US

(73) Assignee: Wyeth, Madison, NJ (US)

(21) Appl. No.: 11/810,911

(22) Filed: Jun. 7, 2007

Related U.S. Application Data
(60) Provisional application No. 60/812,146, filed on Jun. 9, 2006.

(51) Int. Cl. A61K 31/433; C07D 285/14; A61P 35/00; A61P 19/02

(52) U.S. Cl. 514/363; 548/126

(57) ABSTRACT

The present invention relates to Thiadiazole Compounds; compositions comprising an effective dose of a Thiadiazole Compound; and methods treating or preventing a metalloproteinase-related disorder, such as, an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia or a periodontal disease or comprising administering an effective dose of a Thiadiazole Compound to a mammal in need thereof.

This application claims priority to U.S. provisional patent application No. 60/812,146, filed on Jun. 9, 2006, which is hereby incorporated by reference in its entirety.

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

### 1. FIELD OF THE INVENTION

The present invention relates to Thiadiazole Compounds, as defined below; compositions comprising an effective dose of a Thiadiazole Compound; and methods for treating or preventing a metalloproteinase-related disorder comprising administering to a mammal in need thereof an effective dose of a Thiadiazole Compound.

### 2. BACKGROUND OF THE INVENTION

Metalloproteinases, including matrix metalloproteinases and aggrecanases, are known to have a role in the breakdown of connective tissue. Matrix metalloproteinases (“MMPs”) constitute a superfamily of proteolytic enzymes that are genetically related and capable of degrading almost all the constituents of extracellular matrix and basement membrane that restrict cell movement. Aggrecanases are members of the ADAMTS (A disintegrin and metalloproteinase with thrombospondin motifs) family of proteins. Aggrecanase-1 and aggrecanase-2 have been designated ADAMTS-4 and ADAMTS-5, respectively (Tang, Int. J. Biochem. Cell. Biol. 33:33-44 (2001)).

The ADAMTS family is involved in cleaving aggrecan, a cartilage component also known as the large aggregating chondroitin sulphate proteoglycan (Abbaszade et al., J. Biol. Chem. 274:23443-23450 (1999)), procollagen processing (Colige et al., Proc. Natl. Acad. Sci. USA 94:2374-2379 (1997)), angiogenesis and tumor invasion (Vazquez et al., J. Biol. Chem. 274:23349-23357 (1999)), inflammation (Kuno et al., J. Biol. Chem. 272:556-562 (1997)). MMPs have been shown to cleave aggrecan as well.

The loss of aggrecan has been implicated in the degradation of articular cartilage in arthritic diseases, for example osteoarthritis is a debilitating disease which affects at least 30 million Americans. Degradation of articular cartilage and the resulting chronic pain can severely reduce quality of life. An early and important characteristic of the osteoarthritic process is loss of aggrecan from the extracellular matrix, resulting in deficiencies in the biomechanical characteristics of the cartilage. Likewise, MMPs and aggrecanases are known to play a role in many disorders in which extracellular protein degradation or destruction occurs, such as cancer, asthma, chronic obstructive pulmonary disease (“COPD”), atherosclerosis, age-related macular degeneration, myocardial infarction, corneal ulceration and other ocular surface diseases, hepatitis, aortic aneurysms, tendonitis, central nervous system diseases, abnormal wound healing, angiogenesis, restenosis, cirrhosis, multiple sclerosis, glomerulonephritis, graft versus host disease, diabetes, inflammatory bowel disease, shock, invertebral disc degeneration, stroke, osteopenia, and periodontal diseases.

Accordingly, there is a need for metalloproteinase inhibitors, such as inhibitors of MMPs and aggrecanases. The present invention addresses this need.

### 3. SUMMARY OF THE INVENTION

The present invention relates to Thiadiazole Compounds; compositions comprising an effective dose of a Thiadiazole Compound; and methods for treating or preventing a metalloproteinase-related disorder, such as, an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia or a periodontal disease comprising administering an effective dose of a Thiadiazole Compound to a mammal in need thereof.

In one embodiment, the invention provides compounds of the Formula (I):

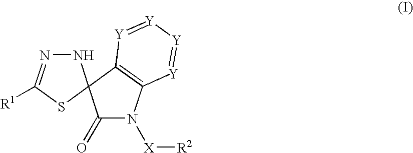

or a pharmaceutically acceptable salt or hydrate thereof,
wherein

X is —(CH₂)ₘ—C(O)O—, —(CH₂)ₘ—C(O)NH— or —(CH₂)ₘ—SO₂—;

m is 0 or 1;

each Y is —C(R³)—;

R¹ is -aryl or -5 or 6-membered aromatic or non-aromatic heterocycle, wherein the -aryl or -5 or 6-membered aromatic or non-aromatic heterocycle group is unsubstituted or substituted with one or more R⁴ groups;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

In one embodiment, the invention provides compounds of the Formula (II):

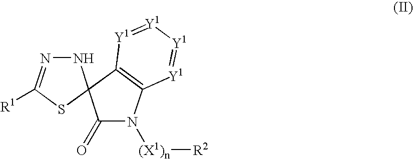

or a pharmaceutically acceptable salt or hydrate thereof,
wherein

X¹ is —CH₂—, —(CH₂)ₘ—C(O)—, —(CH₂)ₘ—C(O)NR²—, —(CH₂)ₘ—C(O)O—, —(CH₂)ₘ—C(O)NH— or —(CH₂)ₘ—SO₂—;

m is 0 or 1;

n is 0 or 1;

each Y¹ is independently —C(R³)— or —N—, wherein at least one occurrence of Y¹ is —N—;

R¹ is -aryl or -5 or 6-membered aromatic or non-aromatic heterocycle, wherein the -aryl or -5 or 6-membered aromatic or non-aromatic heterocycle group is unsubstituted or substituted with one or more R⁴ groups;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

In one embodiment, the invention provides compounds of the Formula (III):

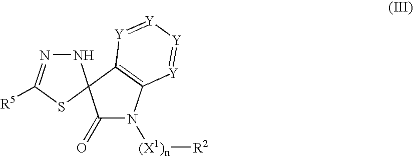

or a pharmaceutically acceptable salt or hydrate thereof,
wherein

X¹ is —CH₂—, —(CH₂)ₘ—C(O)—, —(CH₂)ₘ—C(O)NR²—, —(CH₂)ₘ—C(O)O—, —(CH₂)ₘ—C(O)NH— or —(CH₂)ₘ—SO₂—;

m is 0 or 1;

n is 0 or 1;

each Y is independently —C(R³)— or —N—;

R⁵ is -5 or 6-membered aromatic or non-aromatic heterocycle, which is unsubstituted or substituted with one or more R⁴ groups;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic, or non-aromatic heterocycle or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆, alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

In one embodiment, the invention provides compounds of the Formula (IV):

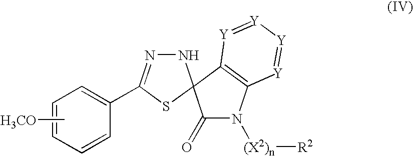

or a pharmaceutically acceptable salt or hydrate thereof,
wherein

X² is —CH₂—, —(CH₂)ₘ—C(O)O—, —(CH₂)ₘ—C(O)NH—, —(CH₂)ₘ—C(O)NR²—, or —(CH₂)ₘ—SO₂—;

each Y is independently —C(R³)— or —N—;

m is 0 or 1;

n is 0 or 1;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂,

wherein the —OCH₃ depicted in Formula (IV) occupies the para position or an ortho or meta position on the phenyl ring to which it is attached.

In one embodiment, the invention provides compounds of the Formula (V):

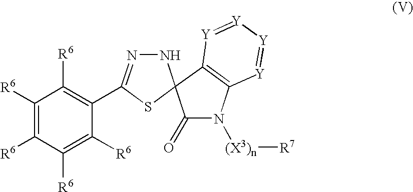

or a pharmaceutically acceptable salt or hydrate thereof,
wherein

X³ is —CH₂—, —(CH₂)ₘ—C(O)O—, —(CH₂)ₘ—C(O)NH—, or —(CH₂)ₘ—SO₂—;

each Y is independently —C(R⁸)— or —N—;

m is 0 or 1;

n is 0 or 1;

each R⁶ is independently —H, —C₁-C₆ alkyl, or -halo;

R⁷ is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein a —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), such that when n is 0, R⁷ is not —H; and

each R⁸ is independently —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

In one embodiment, the invention provides compounds of the Formula (VI):

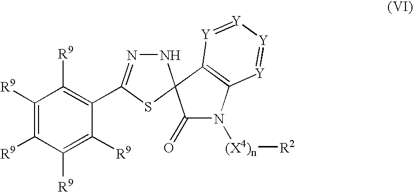

or a pharmaceutically acceptable salt or hydrate thereof,
wherein

X⁴ is —CH₂—, —C(O)—, —(CH₂)ₘ—C(O)O—, —(CH₂)ₘ—C(O)NH, or —(CH₂)ₘ—SO₂—;

each Y is independently —C(R³)— or —N—;

m is 0 or 1;

n is 0 or 1;

each R⁹ is independently —H, —C₁-C₆ alkyl, —O—C₁-C₆ alkyl, or -halo;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂,

such that:

every R⁹ and every R³ are not simultaneously —H when n is 0 and R² is —H, methyl, ethyl, butyl, pentyl, or an unsubstituted or mono- or di-substituted —(C₁-C₆ alkyl)-aryl;

R⁹ and R² are not simultaneously —H when n is 0, exactly one R³ group is bromo, isopropyl, ethyl, or methyl and the other R³ groups are —H;

R² is not naphthyl, methyl, butyl, or pentyl when every R⁹ and R³ group is —H and n is 0;

R² is not —H or methyl when n is 0, exactly one R⁹ group is methyl while the other R⁹ groups are —H, and every R³ group is —H or exactly one R³ group is methyl or ethyl while the other R³ groups are —H;

R² is not naphthyl substituted with exactly one -halo or one —NO₂ group, or phenyl substituted with exactly one -halo or one —NO₂ group, when (X)ₙ is —CH₂— and every R⁹ and R³ group is —H or exactly one R⁹ group is methyl and the other R⁹ and R³ groups are —H;

R² is not —H, methyl, or —(C₁-C₆ alkyl)-(5 or 6-membered non-aromatic heterocycle) when exactly one R⁹ group is methyl, n is 0, and every R³ group is —H;

X⁴ is not —C(O)— and R² is not propyl or methyl when every R³ is —H, n is 1, and every R⁹ is —H or exactly one R⁹ is —O—(C₁-C₆ alkyl) and the other R⁹ groups are —H;

exactly one R³ group is not methyl, ethyl, or isopropyl when the other three R³ groups are —H, and each R⁹ group is —H or the R⁹ group at the para position is methyl and the other R⁹ groups are —H, and R² is —H; and

the R³ groups do not comprise exactly one methyl and exactly one -halo or the R³ groups do not comprise exactly two methyl groups at the 6 and 7 positions of the indoline ring when n is 0 and R² and every R⁹ are —H.

In one embodiment, the invention provides compounds of the Formula (VII):

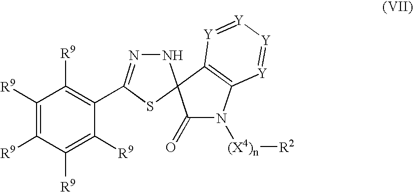

or a pharmaceutically acceptable salt or hydrate thereof,
wherein

X⁴ is —CH₂—, —C(O)—, —(CH₂)ₘ—C(O)O—, —(CH₂)ₘ—C(O)NH, —(CH₂)ₘ—C(O)NR², or —(CH₂)ₘ—SO₂—;

each Y is independently —C(R³)— or —N—;

m is 0 or 1;

n is 0 or 1;

each R⁹ is independently —H, —C₁-C₆ alkyl, —O—C₁-C₆ alkyl, or halo;

R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and

each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

A compound of Formula (I), (II), (III), (IV), (V), (VI) or (VII), or a pharmaceutically acceptable salt or hydrate thereof, (hereinafter, each individually or collectively being a “Thiadiazole Compound”) is useful for treating or preventing an arthritic disorder, osteoarthritis, cancer, rheumatoid arthritis, asthma, chronic obstructive pulmonary disease, atherosclerosis, age-related macular degeneration, a myocardial infarction, a corneal ulceration, an ocular surface disease, hepatitis, an aortic aneurysm, tendonitis, a central nervous system disorder, a wound that has healed abnormally, angiogenesis, restenosis, cirrhosis, multiple sclerosis, glomerulonephritis, graft versus host disease, diabetes, an inflammatory bowel disease, shock, invertebral disc degeneration, stroke, osteopenia, a periodontal disease or a metalloproteinase-related disorder (hereinafter, each individually or collectively being a “Condition”).

The invention also provides compositions comprising an effective dose of a Thiadiazole Compound and a physiologically acceptable carrier or vehicle. The compositions are useful for treating or preventing a Condition.

The invention further provides methods for treating or preventing a Condition comprising administering an effective dose of a Thiadiazole Compound to a mammal in need thereof.

The details of the invention are set forth in the accompanying description below. Other features, objects, and advantages of the invention will be apparent from the description and from the claims. All patents, patent applications and publications cited in this specification are incorporated herein by reference in their entireties for all purposes.

### 4. DETAILED DESCRIPTION OF THE INVENTION

### 4.1 Definitions and Abbreviations

The term “C₁-C₆ alkyl” as used herein refers to a straight or branched chain, saturated hydrocarbon having from 1 to 6 carbon atoms. Representative C₁-C₆ alkyl groups include, but are not limited to methyl, ethyl, n-propyl, isopropyl, n-butyl, sec-butyl, tert-butyl, n-pentyl, isopentyl, neopentyl, n-hexyl, isohexyl, and neohexyl. Unless indicated, the C₁-C₆ alkyl group is unsubstituted.

The term “C₂-C₆ alkenyl” refers to a straight or branched chain hydrocarbon containing 2-6 carbon atoms and at least one carbon-carbon double bond. Representative C₂-C₆ alkenyl groups include, but are not limited to, ethylenyl, 1-propylenyl, 2-propylenyl, 1-butylenyl, 2-butylenyl, isobutylenyl, sec-butylenyl, 1-pentenyl, 2-pentenyl, isopentenyl, 1-hexenyl, 2-hexenyl, 3-hexenyl and isohexenyl. In one embodiment, the C₂-C₆ alkenyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted C₁-C₆ alkyl. Unless indicated, the C₂-C₆ alkenyl group is unsubstituted. In one embodiment, “C₂-C₆ alkenyl” does not include allyl.

The term “C₂-C₆ alkynyl” refers to a straight or branched chain hydrocarbon containing 2-6 carbon atoms and at least one carbon-carbon triple bond. Representative C₂-C₆ alkynyl groups include, but are not limited to, acetylenyl, 1-propynyl, 2-propynyl, 2-butynyl, isobutynyl, sec-butynyl, 1-pentynyl, 2-pentynyl, isopentynyl, 1-hexynyl, 2-hexynyl, 3-hexynyl and isohexynyl. In one embodiment, the C₂-C₆ alkynyl group is substituted with one or more of the following groups: -halo, —O—(C₁-C₆ alkyl), —OH, —CN, —COOR′, —OC(O)R′, —N(R′)₂, —NHC(O)R′ or —C(O)NHR′ groups wherein each R′ is independently —H or unsubstituted —C₁-C₆ alkyl. Unless indicated, the C₂-C₆ alkynyl group is unsubstituted.

The term “aryl” as used herein as a group or part of a group refers to a phenyl or naphthyl group. Unless indicated, the aryl group is unsubstituted.

The term “5- or 6-membered aromatic or non-aromatic heterocycle” refers to a 5- or 6-membered aromatic or non-aromatic monocyclic cycloalkyl in which 1-4 of the ring carbon atoms have been independently replaced with a N, O or S atom. A 5- or 6-membered aromatic or non-aromatic heterocycle is attached via a ring carbon or ring nitrogen atom. In one embodiment, a 5- or 6-membered aromatic or non-aromatic heterocycle is fused to a benzene ring. Representative examples of a 5- or 6-membered aromatic or non-aromatic heterocycle group include, but are not limited to furanyl, imidazolyl, imidazolinyl, imidazolidinyl, pyrazolinyl, pyrazolidinyl, pyranyl, piperidinyl, piperazinyl, dioxolinyl, dioxanyl, morpholinyl, dithianyl, thiomorpholinyl, pyrrolidinyl, pyrrolinyl, isothiazolyl, isoxazolyl, oxadiazolyl, oxazolidinyl, oxazolyl, oxazolidinyl, pyrimidinyl, pyrazinyl, pyrazolyl, pyridazinyl, pyridinyl, thiazolyl, thiadiazolyl, thiophenyl, triazinyl, triazolyl. Unless indicated, the 5- or 6-membered aromatic or non-aromatic heterocycle group is unsubstituted.

The number of carbon atoms as used in the definitions herein refers only to the number of carbon atoms in the carbon skeleton of the functional group being defined and does not include carbon atoms present in any substituents that may be attached to the functional group being defined.

The term “halo” as used herein refers to —F, —Cl, —Br, or —I.

The term “metalloproteinase-related disorder” used herein refers to a condition for which it would be beneficial to modulate activity of the metalloproteinase. Illustrative metalloproteinase-related disorders include, but are not limited to, an arthritic disorder, osteoarthritis, cancer, rheumatoid arthritis, asthma, chronic obstructive pulmonary disease, atherosclerosis, age-related macular degeneration, myocardial infarction, corneal ulceration, an ocular surface disease, hepatitis, an aortic aneurysm, tendonitis, a central nervous system disease, abnormal wound healing, angiogenesis, restenosis, cirrhosis, multiple sclerosis, glomerulonephritis, a graft versus host disease, diabetes, inflammatory bowel disease, shock, invertebral disc degeneration, stroke, osteopenia, or a periodontal disease.

The term “metalloproteinase modulator” refers to a compound that is capable of modulating the expression of a metalloproteinase. For example, a metalloproteinase modulator may enhance metalloproteinase expression. A metalloproteinase modulator may also be an inhibitor of a metalloproteinase.

The term “isolated and purified” as used herein refers to an isolate that is separate from other components of a reaction mixture or a natural source. In certain embodiments, the isolate contains at least about 50%, at least about 55%, at least about 60%, at least about 65%, at least about 70%, at least about 75%, at least about 80%, at least about 85%, at least about 90%, at least about 95%, or at least about 98% of the compound or pharmaceutically acceptable salt or hydrate of the compound by weight of the isolate.

The term “effective dose” as used herein refers to an amount of a Thiadiazole Compound that is effective for treating or preventing a Condition.

The phrase “pharmaceutically acceptable salt,” as used herein, refers to a salt of an acid and a basic nitrogen atom of a Thiadiazole Compound. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The pharmaceutically acceptable salt can also be a camphorsulfonate salt. The term “pharmaceutically acceptable salt” also refers to a salt of a Thiadiazole Compound having an acidic functional group, such as a carboxylic acid functional group, and a base. Suitable bases include, but are not limited to, hydroxides of alkali metals such as sodium, potassium, and lithium; hydroxides of alkaline earth metals such as calcium and magnesium; hydroxides of other metals, such as aluminum and zinc; ammonia, and organic amines, such as unsubstituted or hydroxy-substituted mono-, di-, or tri-alkylamines, dicyclohexylamine; tributyl amine; pyridine; N-methyl, N-ethylamine; diethylamine; triethylamine; mono-, bis-, or tris-(2-OH-lower alkylamines), such as mono-; bis-, or tris-(2-hydroxyethyl)amine, 2-hydroxy-tert-butylamine, or tris-(hydroxymethyl)methylamine, N,N-di-lower alkyl-N-(hydroxyl-lower alkyl)-amines, such as N,N-dimethyl-N-(2-hydroxyethyl)amine or tri-(2-hydroxyethyl)amine; N-methyl-D-glucamine; and amino acids such as arginine, lysine, and the like. The term “pharmaceutically acceptable salt” may also include a pharmaceutically acceptable salt of a hydrate of a Thiadiazole Compound.

A “mammal” is a human, mouse, rat, guinea pig, dog, cat, horse, cow, pig, or non-human primate, such as a monkey, chimpanzee, baboon or rhesus. In one embodiment, a mammal is a human.

The following abbreviations are used herein and have the indicated definitions: ACN is acetonitrile; CHO is chinese hamster ovary; DMSO is dimethylsulfoxide; EtOH is ethanol; Et₂O is diethyl ether; HEPES is N-(2-hydroxyethyl)-piperazine-N′-(2-ethanesulfonic acid); HPLC is high-performance liquid chromatography; LC/MS is liquid chromatography/mass spectrometry; and RFU is relative fluorescence units.

### 4.2 The Thiadiazole Compounds

### 4.2.1 The Thiadiazole Compounds of Formula (I)

As stated above, the present invention encompasses Thiadiazole Compounds having the Formula (I):

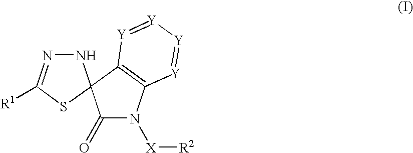

or a pharmaceutically acceptable salt or hydrate thereof, wherein X, Y, R¹ and R² are as defined above for the Thiadiazole Compounds of Formula (I).

In one embodiment, each occurrence of Y is —CH—.

In one embodiment, three occurrences of Y are —CH—.

In one embodiment, three occurrences of Y are —CH— and one occurrence of Y is —C(R³)—.

In one embodiment, X is —(CH₂)ₘ—C(O)O—.

In another embodiment, X is —(CH₂)ₘ—C(O)NH—.

In still another embodiment, X is —(CH₂)ₘ—SO₂—.

In one embodiment, R¹ is aryl.

In one embodiment, R¹ is phenyl.

In one embodiment, R1 is phenyl, substituted with one or more —O—(C₁-C₆ alkyl) groups.

In another embodiment, R¹ is 2-methoxyphenyl.

In still another embodiment, R¹ is 3-methoxyphenyl.

In yet another embodiment, R¹ is 4-methoxyphenyl.

In one embodiment, R¹ is phenyl, substituted with one or more —C₁-C₆ alkyl groups.

In one embodiment, R¹ is phenyl, substituted with one or more methyl groups.

In one embodiment, R¹ is phenyl, substituted with one isopropyl group.

In another embodiment, R¹ is 3-methylphenyl.

In another embodiment, R¹ is 4-methylphenyl.

In another embodiment, R¹ is 4-isopropylphenyl.

In one embodiment, R¹ is phenyl, substituted with one or more halo groups.

In one embodiment, R¹ is phenyl, substituted with one fluoro group.

In one embodiment, R¹ is phenyl, substituted with one chloro group.

In one embodiment, R¹ is 4-fluorophenyl.

In another specific embodiment, R¹ is naphthyl.

In one embodiment, R² is —H.

In another embodiment, R² is —C₁-C₆ alkyl.

In a specific embodiment, R² is methyl.

In another specific embodiment, R² is ethyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —(CH₂)ₘC(O)O—(C₁-C₆ alkyl).

In another embodiment, R² is —C₂-C₆ alkenyl.

In another embodiment, R² is —C₂-C₆ alkynyl.

In a specific embodiment, R² is —CH₂—C≡CH.

In another embodiment, R² is -aryl.

In a specific embodiment, R² is -phenyl.

In another embodiment, R² is —(C₁-C₆ alkyl)-aryl.

In a specific embodiment, R² is -benzyl.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, at least one occurrence of R³ is -halo.

In a specific embodiment, at least one occurrence of R³ is —Cl.

In another specific embodiment, at least one occurrence of R³ is —Br.

In another specific embodiment, at least one occurrence of R³ is —F.

In one embodiment, at least one occurrence of R³ is —O—(C₁-C₆ alkyl).

In a specific embodiment, at least one occurrence of R³ is —OCH₃.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In a specific embodiment, at least one occurrence of R³ is —CF₃.

In another specific embodiment, at least one occurrence of R³ is —OCF₃.

In yet another specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, one occurrence of R³ is C₁-C₆ alkyl and another occurrence of R³ is halo.

In another embodiment, one occurrence of R³ is 5 or 6-membered non-aromatic heterocycle and another occurrence of R³ is halo.

In one embodiment, a compound of Formula (I) is in isolated and purified form.

### 4.2.2 The Thiadiazole Compounds of Formula (II)

As stated above, the present invention encompasses Thiadiazole Compounds having the Formula (II):

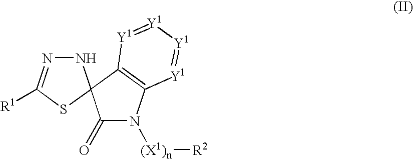

or a pharmaceutically acceptable salt or hydrate thereof, wherein X¹, Y¹, n, R¹ and R² are as defined above for the Thiadiazole Compounds of Formula (II).

In one embodiment, three occurrences of Y¹ are —C(R³)— and one occurrence of Y¹ is —N—.

In another embodiment, two occurrences of Y¹ are —C(R³)— and two occurrences of Y¹ are —N—.

In yet another embodiment, one occurrence of Y¹ are —C(R³)— and three occurrences of Y¹ are —N—.

In one embodiment, each occurrence of Y¹ is —N—.

In one embodiment, each occurrence of —C(R³)— is —CH—.

In one embodiment, X¹ is —(CH₂)ₘ—C(O)O—.

In another embodiment, X¹ is —(CH₂)ₘ—C(O)NH—.

In still another embodiment, X¹ is —CH₂—.

In yet another embodiment, X¹ is —(CH₂)ₘ—C(O)—.

In a further embodiment, X¹ —(CH₂)ₘ—SO₂—.

In another embodiment, X¹ is —(CH₂)ₘ—C(O)NR²—.

In one embodiment, m is 1.

In another embodiment, m is 0.

In one embodiment, n is 1.

In another embodiment, n is 0.

In one embodiment, R¹ is aryl.

In one embodiment, R¹ is phenyl.

In one embodiment, R¹ is phenyl, substituted with one or more —O—(C₁-C₆ alkyl) groups.

In another embodiment, R¹ is 2-methoxyphenyl.

In still another embodiment, R¹ is 3-methoxyphenyl.

In yet another embodiment, R¹ is 4-methoxyphenyl.

In one embodiment, R¹ is phenyl, substituted with one or more —C₁-C₆ alkyl groups.

In one embodiment, R¹ is phenyl, substituted with one or more methyl groups.

In one embodiment, R¹ is phenyl, substituted with one isopropyl group.

In another embodiment, R¹ is 3-methylphenyl.

In another embodiment, R¹ is 4-methylphenyl.

In another embodiment, R¹ is 4-isopropylphenyl.

In one embodiment, R¹ is phenyl, substituted with one or more halo groups.

In one embodiment, R¹ is phenyl, substituted with one fluoro group.

In one embodiment, R¹ is phenyl, substituted with one chloro group.

In one embodiment, R¹ is 4-fluorophenyl.

In another specific embodiment, R¹ is naphthyl.

In one embodiment, R² is —H.

In another embodiment, R² is —C₁-C₆ alkyl.

In a specific embodiment, R² is methyl.

In another specific embodiment, R² is ethyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —C(O)O—(C₁-C₆ alkyl).

In another embodiment, R² is —C₂-C₆ alkenyl.

In another embodiment, R² is —C₂-C₆ alkynyl.

In a specific embodiment, R² is —CH₂—C≡CH.

In another embodiment, R² is -aryl.

In a specific embodiment, R² is -phenyl.

In another embodiment, R² is —(C₁-C₆ alkyl)-aryl.

In a specific embodiment, R² is -benzyl.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, at least one occurrence of R³ is -halo.

In a specific embodiment, at least one occurrence of R³ is —Cl.

In another specific embodiment, at least one occurrence of R³ is —Br.

In another specific embodiment, at least one occurrence of R³ is —F.

In one embodiment, at least one occurrence of R³ is —O—(C₁-C₆ alkyl).

In a specific embodiment, at least one occurrence of R³ is —OCH₃.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In a specific embodiment, at least one occurrence of R³ is —CF₃.

In another specific embodiment, at least one occurrence of R³ is —OCF₃.

In yet another specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, one occurrence of R³ is C₁-C₆ alkyl and another occurrence of R³ is halo.

In another embodiment, one occurrence of R³ is 5 or 6-membered non-aromatic heterocycle and another occurrence of R³ is halo.

In one embodiment, at least one occurrence of R⁴ is -halo.

In a specific embodiment, at least one occurrence of R⁴ is —Cl.

In another specific embodiment, at least one occurrence of R⁴ is —Br.

In another specific embodiment, at least one occurrence of R⁴ is —F.

In one embodiment, at least one occurrence of R⁴ is —O—(C₁-C₆ alkyl).

In a specific embodiment, at least one occurrence of R⁴ is —OCH₃.

In one embodiment, at least one occurrence of R⁴ is —C₁-C₆ alkyl.

In a specific embodiment, at least one occurrence of R⁴ is methyl.

In another specific embodiment, at least one occurrence of R⁴ is ethyl.

In yet another specific embodiment, at least one occurrence of R⁴ is n-propyl.

In another specific embodiment, at least one occurrence of R⁴ is isopropyl.

In a specific embodiment, at least one occurrence of R⁴ is —CF₃.

In another specific embodiment, at least one occurrence of R⁴ is —OCF₃.

In yet another specific embodiment, at least one occurrence of R⁴ is —NO₂.

In one embodiment, at least one occurrence of R⁴ is -5 or 6-membered aromatic or non-aromatic heterocycle.

In a specific embodiment, at least one occurrence of R⁴ is piperazin-1-yl.

In one embodiment, at least one occurrence of R⁴ is —(C₁-C₆ alkyl)-(5 or 6-membered aromatic or non-aromatic heterocycle).

In one embodiment, at least one occurrence of R⁴ is -(5 or 6-membered aromatic or non-aromatic heterocycle)-aryl.

In one embodiment, at least one occurrence of R⁴ is -(5 or 6-membered aromatic or non-aromatic heterocycle)-CH₂-aryl.

In one embodiment, two occurrences of R⁴ are independently C₁-C₆ alkyl.

In a specific embodiment, two occurrences of R⁴ are methyl.

In one embodiment, one occurrence of R⁴ is C₁-C₆ alkyl and another occurrence of R⁴ is halo.

In another embodiment, one occurrence of R⁴ is 5 or 6-membered non-aromatic heterocycle and another occurrence of R⁴ is halo.

In one embodiment, a compound of Formula (II) is in isolated and purified form.

### 4.2.3 The Thiadiazole Compounds of Formula (III)

As stated above, the present invention encompasses Thiadiazole Compounds having the Formula (III):

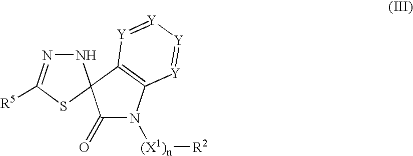

or a pharmaceutically acceptable salt or hydrate thereof, wherein X¹, Y, n, R⁵ and R² are as defined above for the Thiadiazole Compounds of Formula (III).

In one embodiment, three occurrences of Y are independently —C(R³)— and one occurrence of Y is —N—.

In another embodiment, two occurrences of Y are independently —C(R³)— and two occurrences of Y are —N—.

In yet another embodiment, one occurrence of Y is —C(R³)— and three occurrences of Y are —N—.

In one embodiment, each occurrence of Y is —N—.

In one embodiment, each occurrence of Y is independently —C(R³)—.

In a specific embodiment, each occurrence of Y is —CH—.

In one embodiment, each occurrence of —C(R³)— is —CH—.

In one embodiment, X¹ is —(CH₂)ₘ—C(O)—.

In another embodiment, X¹ is —CH₂—.

In still another embodiment, X¹ is —(CH₂)ₘ—C(O)NR²

In one embodiment, n is 1.

In another embodiment, n is 0.

In one embodiment, R² is —H.

In another embodiment, R² is —C₁-C₆ alkyl.

In a specific embodiment, R² is methyl.

In another specific embodiment, R² is ethyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —(CH₂)ₘC(O)O—(C₁-C₆ alkyl).

In another embodiment, R² is —C₂-C₆ alkenyl.

In another embodiment, R² is —C₂-C₆ alkynyl.

In a specific embodiment, R² is —CH₂—C≡CH.

In another embodiment, R² is -aryl.

In a specific embodiment, R² is -phenyl.

In another embodiment, R² is —(C₁-C₆ alkyl)-aryl.

In a specific embodiment, R² is -benzyl.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, at least one occurrence of R³ is -halo.

In a specific embodiment, at least one occurrence of R³ is —Cl.

In another specific embodiment, at least one occurrence of R³ is —Br.

In another specific embodiment, at least one occurrence of R³ is —F.

In one embodiment, at least one occurrence of R³ is —O—(C₁-C₆ alkyl).

In a specific embodiment, at least one occurrence of R³ is —OCH₃.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In a specific embodiment, at least one occurrence of R³ is —CF₃.

In another specific embodiment, at least one occurrence of R³ is —OCF₃.

In yet another specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, one occurrence of R³ is C₁-C₆ alkyl and another occurrence of R³ is halo.

In another embodiment, one occurrence of R³ is 5 or 6-membered non-aromatic heterocycle and another occurrence of R³ is halo.

In one embodiment, at least one occurrence of R⁴ is -halo.

In a specific embodiment, at least one occurrence of R⁴ is —Cl.

In another specific embodiment, at least one occurrence of R⁴ is —Br.

In another specific embodiment, at least one occurrence of R⁴ is —F.

In one embodiment, at least one occurrence of R⁴ is —O—(C₁-C₆ alkyl).

In a specific embodiment, at least one occurrence of R⁴ is —OCH₃.

In one embodiment, at least one occurrence of R⁴ is —C₁-C₆ alkyl.

In a specific embodiment, at least one occurrence of R⁴ is methyl.

In another specific embodiment, at least one occurrence of R⁴ is ethyl.

In yet another specific embodiment, at least one occurrence of R⁴ is n-propyl.

In another specific embodiment, at least one occurrence of R⁴ is isopropyl.

In a specific embodiment, at least one occurrence of R⁴ is —CF₃.

In another specific embodiment, at least one occurrence of R⁴ is —OCF₃.

In yet another specific embodiment, at least one occurrence of R⁴ is —NO₂.

In one embodiment, at least one occurrence of R⁴ is -5 or 6-membered aromatic or non-aromatic heterocycle.

In a specific embodiment, at least one occurrence of R⁴ is piperazin-1-yl.

In one embodiment, at least one occurrence of R⁴ is —(C₁-C₆ alkyl)-(5 or 6-membered aromatic or non-aromatic heterocycle).

In one embodiment, at least one occurrence of R⁴ is -(5 or 6-membered aromatic or non-aromatic heterocycle)-aryl.

In one embodiment, at least one occurrence of R⁴ is -(5 or 6-membered aromatic or non-aromatic heterocycle)-CH₂-aryl.

In one embodiment, two occurrences of R⁴ are independently C₁-C₆ alkyl.

In a specific embodiment, two occurrences of R⁴ are methyl.

In one embodiment, one occurrence of R⁴ is C₁-C₆ alkyl and another occurrence of R⁴ is halo.

In another embodiment, one occurrence of R⁴ is 5 or 6-membered non-aromatic heterocycle and another occurrence of R⁴ is halo.

In one embodiment, R⁵ is 5-membered aromatic heterocycle;

In one embodiment, R⁵ is 6-membered aromatic heterocycle;

In one embodiment, R⁵ is 5-membered non-aromatic heterocycle;

In one embodiment, R⁵ is 6-membered non-aromatic heterocycle;

In one embodiment, a compound of Formula (III) is in isolated and purified form.

### 4.2.4 The Thiadiazole Compounds of Formula (IV)

As stated above, the present invention encompasses Thiadiazole Compounds having the Formula (IV):

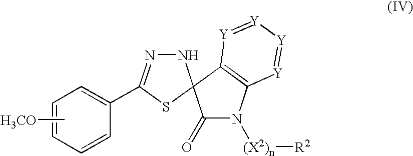

or a pharmaceutically acceptable salt or hydrate thereof, wherein X², Y, n, and R² are as defined above for the Thiadiazole Compounds of Formula (IV), and wherein the —OCH₃ depicted in Formula (IV) occupies the para position or an ortho or meta position on the phenyl ring to which it is attached.

In one embodiment, three occurrences of Y are independently —C(R³)— and one occurrence of Y is —N—.

In another embodiment, two occurrences of Y are independently —C(R³)— and two occurrences of Y are —N—.

In yet another embodiment, one occurrence of Y is —C(R³)— and three occurrences of Y are —N—.

In one embodiment, each occurrence of Y is —N—.

In one embodiment, each occurrence of Y is independently —C(R³)—.

In a specific embodiment, each occurrence of Y is —CH—.

In one embodiment, each occurrence of —C(R³)— is —CH—.

In one embodiment, X² is —(CH₂)ₘ—C(O)O—.

In another embodiment, X² is —(CH₂)ₘ—C(O)NH—.

In still another embodiment, X² is —CH₂—.

In a further embodiment, X² is —(CH₂)ₘ—SO₂—.

In another embodiment, X² is —(CH₂)ₘ—C(O)NR²—.

In one embodiment, n is 1.

In another embodiment, n is 0.

In one embodiment, the —OCH₃ depicted in Formula (IV) occupies the para position on the phenyl ring to which it is attached.

In another embodiment, the —OCH₃ depicted in Formula (IV) occupies an ortho position on the phenyl ring to which it is attached.

In still another embodiment, the —OCH₃ depicted in Formula (IV) occupies a meta position on the phenyl ring to which it is attached.

In one embodiment, R² is —H.

In another embodiment, R² is —C₁-C₆ alkyl.

In a specific embodiment, R² is methyl.

In another specific embodiment, R² is ethyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —(CH₂)ₘC(O)O—(C₁-C₆ alkyl).

In another embodiment, R² is —C₂-C₆ alkenyl.

In another embodiment, R² is —C₂-C₆ alkynyl.

In a specific embodiment, R² is —CH₂—C≡CH.

In another embodiment, R² is -aryl.

In a specific embodiment, R² is -phenyl.

In another embodiment, R² is —(C₁-C₆ alkyl)-aryl.

In a specific embodiment, R² is -benzyl.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, at least one occurrence of R³ is -halo.

In a specific embodiment, one occurrence of R³ is —Cl.

In another specific embodiment, one occurrence of R³ is —Br.

In another specific embodiment, one occurrence of R³ is —F.

In one embodiment, at least one occurrence of R³ is —O—(C₁-C₆ alkyl).

In a specific embodiment, one occurrence of R³ is —OCH₃.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In a specific embodiment, at least one occurrence of R³ is —CF₃.

In another specific embodiment, at least one occurrence of R³ is —OCF₃.

In yet another specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, one occurrence of R³ is C₁-C₆ alkyl and another occurrence of R³ is halo.

In another embodiment, one occurrence of R³ is 5 or 6-membered non-aromatic heterocycle and another occurrence of R³ is halo.

In one embodiment, a compound of Formula (IV) is in isolated and purified form.

Illustrative examples of compounds of Formula (IV) are those compounds set forth below:

| Compound | Name |
|---|---|
| 1 | 5′-(4-methoxyphenyl)-5,7-dimethyl-3′H-spiro[indole-3,2′- |
| | [1,3,4]thiadiazol]-2(1H)-one |
| 2 | 5′-(4-methoxyphenyl)-1-methyl-3′H-spiro[indole-3,2′-[1,3,4]thiadiazol]- |
| | 2(1H)-one |
| 3 | 5′-(4-methoxyphenyl)-3′H-spiro[indole-3,2′-[1,3,4]thiadiazol]-2(1H)-one |
| 4 | 5-bromo-5′-(4-methoxyphenyl)-3′H-spiro[indole-3,2′-[1,3,4]thiadiazol]- |
| | 2(1H)-one |
| 5 | 5′-(4-methoxyphenyl)-5-methyl-3′H-spiro[indole-3,2′-[1,3,4]thiadiazol]- |
| | 2(1H)-one |
| 6 | 5-methoxy-5′-(4-methoxyphenyl)-3′H-spiro[indole-3,2′-[1,3,4]thiadiazol]- |
| | 2(1H)-one |
| 7 | 5′-(4-methoxyphenyl)-1-prop-2-yn-1-yl-3′H-spiro[indole-3,2′- |
| | [1,3,4]thiadiazol]-2(1H)-one |
| 8 | 5′-(4-methoxyphenyl)-1-[3-(trifluoromethyl)phenyl]-3′H-spiro[indole- |
| | 3,2′-[1,3,4]thiadiazol]-2(1H)-one |
| 9 | ethyl [5′-(4-methoxyphenyl)-2-oxo-3′H-spiro[indole-3,2′- |
| | [1,3,4]thiadiazol]-1(2H)-yl]acetate |
| 10 | 1-benzyl-5′-(4-methoxyphenyl)-3′H-spiro[indole-3,2′-[1,3,4]thiadiazol]- |
| | 2(1H)-one |
| 11 | 1-[2-(dimethylamino)ethyl]-5′-(4-methoxyphenyl)-3′H-spiro[indole-3,2′- |
| | [1,3,4]thiadiazol]-2(1H)-one |
| 12 | 6-chloro-5′-(4-methoxyphenyl)-3′H-spiro[indole-3,2′-[1,3,4]thiadiazol]- |
| | 2(1H)-one |
| 13 | 5-chloro-5′-(4-methoxyphenyl)-3′H-spiro[indole-3,2′-[1,3,4]thiadiazol]- |
| | 2(1H)-one |
| 14 | 4-(4-benzylpiperazin-1-yl)-5′-(4-methoxyphenyl)-3′H-spiro[indole-3,2′- |
| | [1,3,4]thiadiazol]-2(1H)-one |
| 15 | 5′-(4-methoxyphenyl)-6-propyl-3′H-spiro[indole-3,2′-[1,3,4]thiadiazol]- |
| | 2(1H)-one |
| 16 | 5′-(4-methoxyphenyl)-5-nitro-3′H-spiro[indole-3,2′-[1,3,4]thiadiazol]- |
| | 2(1H)-one |
| 17 | 6-chloro-5′-(4-methoxyphenyl)-7-methyl-3′H-spiro[indole-3,2′- |
| | [1,3,4]thiadiazol]-2(1H)-one |
| 18 | 5′-(4-methoxyphenyl)-7-methyl-3′H-spiro[indole-3,2′-[1,3,4]thiadiazol]- |
| | 2(1H)-one |
| 19 | 5′-(4-methoxyphenyl)-6-methyl-3′H-spiro[indole-3,2′-[1,3,4]thiadiazol]- |
| | 2(1H)-one |
| 20 | 7-methoxy-5′-(4-methoxyphenyl)-6-methyl-3′H-spiro[indole-3,2′- |
| | [1,3,4]thiadiazol]-2(1H)-one |
| 21 | 1-ethyl-5-fluoro-5′-(4-methoxyphenyl)-6-(4-methylpiperazin-1-yl)-3′H- |
| | spiro[indole-3,2′-[1,3,4]thiadiazol]-2(1H)-one |
| 22 | 1-benzyl-5′-(4-methoxyphenyl)-5-methyl-3′H-spiro[indole-3,2′- |
| | [1,3,4]thiadiazol]-2(1H)-one |
| 23 | 5′-(4-methoxyphenyl)-5-methyl-1-(2-methylphenyl)-3′H-spiro[indole- |
| | 3,2′-[1,3,4]thiadiazol]-2(1H)-one |
| 24 | 5′-(4-methoxyphenyl)-5-methyl-1-(4-methylphenyl)-3′H-spiro[indole- |
| | 3,2′-[1,3,4]thiadiazol]-2(1H)-one |
| 25 | 1-(4-chlorophenyl)-5′-(4-methoxyphenyl)-5-methyl-3′H-spiro[indole-3,2′- |
| | [1,3,4]thiadiazol]-2(1H)-one |
| 26 | 1-(3,4-dichlorophenyl)-5′-(4-methoxyphenyl)-5-methyl-3′H-spiro[indole- |
| | 3,2′-[1,3,4]thiadiazol]-2(1H)-one |
| 27 | 5′-(4-methoxyphenyl)-5-methyl-1-(3-nitrophenyl)-3′H-spiro[indole-3,2′- |
| | [1,3,4]thiadiazol]-2(1H)-one |
| 28 | 1-benzyl-5′-(4-methoxyphenyl)-6-propyl-3′H-spiro[indole-3,2′- |
| | [1,3,4]thiadiazol]-2(1H)-one |
| 29 | 5-ethyl-5′-(4-methoxyphenyl)-3′H-spiro[indole-3,2′-[1,3,4]thiadiazol]- |
| | 2(1H)-one |
| 30 | 5-ethyl-5′-(3-methoxyphenyl)-3′H-spiro[indole-3,2′-[1,3,4]thiadiazol]- |
| | 2(1H)-one |

and pharmaceutically acceptable salts or hydrates thereof.

### 4.2.5 The Thiadiazole Compounds of Formula (V)

As stated above, the present invention encompasses Thiadiazole Compounds having the Formula (V):

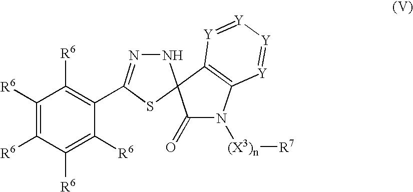

or a pharmaceutically acceptable salt or hydrate thereof, wherein X³, Y, n, R⁶ and R⁷ are as defined above for the Thiadiazole Compounds of Formula (V).

In one embodiment, three occurrences of Y are independently —C(R⁸)— and one occurrence of Y is —N—.

In another embodiment, two occurrences of Y are independently —C(R⁸)— and two occurrences of Y are —N—.

In yet another embodiment, one occurrence of Y is —C(R⁸)— and three occurrences of Y are —N—.

In one embodiment, each occurrence of Y is —N—.

In one embodiment, each occurrence of Y is independently —C(R⁸)—.

In one embodiment, X³ is —(CH₂)ₘ—C(O)O—.

In another embodiment, X³ is —(CH₂)ₘ—C(O)NH—.

In still another embodiment, X³ is —CH₂—.

In a further embodiment, X³ is —(CH₂)ₘ—SO₂—.

In one embodiment, n is 1.

In another embodiment, n is 0.

In one embodiment, m is 1.

In another embodiment, m is 0.

In one embodiment, each occurrence of R⁶ is —H.

In another embodiment, four occurrences of R⁶ are —H.

In still another embodiment, three occurrences of R⁶ are —H.

In one embodiment, one occurrence of R⁶ is C₁-C₆ alkyl.

In another embodiment, two occurrence of R⁶ are C₁-C₆ alkyl.

In still another embodiment, one occurrence of R⁶ is methyl.

In yet another embodiment, one occurrence of R⁶ is isopropyl.

In one embodiment, R⁶ is -halo.

In another embodiment, one occurrence of R⁶ is —F.

In one embodiment, R⁶ is in the para position on the phenyl ring to which it is attached.

In another embodiment, R⁶ is in an ortho position on the phenyl ring to which it is attached.

In still another embodiment, R⁶ is in a meta position on the phenyl ring to which it is attached.

In one embodiment, R⁷ is H.

In another embodiment, R⁷ is not H.

In yet another embodiment, R⁷ is —C₁-C₆ alkyl.

In a specific embodiment, R⁷ is methyl.

In another specific embodiment, R⁷ is ethyl.

In one embodiment, R⁷ is —(C₁-C₆ alkyl)-N(C₁-C₆ alkyl)₂.

In a specific embodiment, R⁷ is —(CH₂)₂N(CH₃)₂.

In one embodiment, R⁷ is —(CH₂)ₘC(O)O—(C₁-C₆ alkyl).

In another embodiment, R⁷ is —C₂-C₆ alkenyl.

In another embodiment, R⁷ is —C₂-C₆ alkynyl.

In a specific embodiment, R⁷ is —CH₂—C≡CH.

In another embodiment, R⁷ is -aryl.

In a specific embodiment, R⁷ is -phenyl.

In another embodiment, R⁷ is —(C₁-C₆ alkyl)-aryl.

In a specific embodiment, R⁷ is -benzyl.

In another embodiment, R⁷ is —(C₁-C₆ alkyl)-(5 or 6-membered aromatic or non-aromatic heterocycle).

In another embodiment, R⁷ is -5 or 6-membered aromatic or non-aromatic heterocycle.

In one embodiment, at least one occurrence of R⁸ is —C₁-C₆ alkyl.

In a specific embodiment, at least one occurrence of R⁸ is methyl.

In another specific embodiment, at least one occurrence of R⁸ is ethyl.

In yet another specific embodiment, at least one occurrence of R⁸ is n-propyl.

In another specific embodiment, at least one occurrence of R⁸ is isopropyl.

In a specific embodiment, at least one occurrence of R⁸ is —CF₃.

In another specific embodiment, at least one occurrence of R⁸ is —OCF₃.

In yet another specific embodiment, at least one occurrence of R⁸ is —NO₂.

In one embodiment, at least one occurrence of R⁸ is -5 or 6-membered aromatic or non-aromatic heterocycle.

In a specific embodiment, at least one occurrence of R⁸ is piperazin-1-yl.

In one embodiment, at least one occurrence of R⁸ is —(C₁-C₆ alkyl)-(5 or 6-membered aromatic or non-aromatic heterocycle).

In one embodiment, at least one occurrence of R⁸ is -(5 or 6-membered aromatic or non-aromatic heterocycle)-aryl.

In one embodiment, at least one occurrence of R⁸ is -(5 or 6-membered aromatic or non-aromatic heterocycle)-CH₂-aryl.

In one embodiment, two occurrences of R⁸ are independently —C₁-C₆ alkyl.

In a specific embodiment, two occurrences of R⁸ are methyl.

In one embodiment, a compound of Formula (V) is in isolated and purified form.

### 4.2.6 The Thiadiazole Compounds of Formula (VI)

As stated above, the present invention encompasses Thiadiazole Compounds having the Formula (VI):

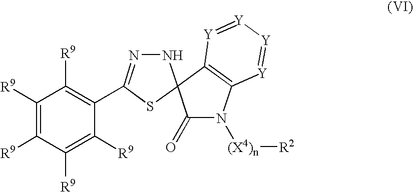

or a pharmaceutically acceptable salt or hydrate thereof, wherein X⁴, Y, n, R⁹ and R² are as defined above for the Thiadiazole Compounds of Formula (VI); such that:

every R⁹ and every R³ are not simultaneously —H when n is 0 and R² is —H, methyl, ethyl, butyl, pentyl, or an unsubstituted or mono- or di-substituted —(C₁-C₆ alkyl)-aryl;

R⁹ and R² are not simultaneously —H when n is 0, exactly one R³ group is bromo, isopropyl, ethyl, or methyl and the other R³ groups are —H;

R² is not naphthyl, methyl, butyl, or pentyl when every R⁹ and R³ group is —H and n is 0;

R² is not —H or methyl when n is 0, exactly one R⁹ group is methyl while the other R⁹ groups are —H, and every R³ group is —H or exactly one R³ group is methyl or ethyl while the other R³ groups are —H;

R² is not naphthyl substituted with exactly one -halo or one —NO₂ group, or phenyl substituted with exactly one -halo or one —NO₂ group, when (X)ₙ is —CH₂— and every R⁹ and R³ group is —H or exactly one R⁹ group is methyl and the other R⁹ and R³ groups are —H;

R² is not —H, methyl, or —(C₁-C₆ alkyl)-(5 or 6-membered non-aromatic heterocycle) when exactly one R⁹ group is methyl, n is 0, and every R³ group is —H;

X⁴ is not —C(O)— and R² is not propyl or methyl when every R³ is —H, n is 1, and every R⁹ is —H or exactly one R⁹ is —O—(C₁-C₆ alkyl) and the other R⁹ groups are —H;

exactly one R³ group is not methyl, ethyl, or isopropyl when the other three R³ groups are —H, and each R⁹ group is —H or the R⁹ group at the para position is methyl and the other R⁹ groups are —H, and R² is —H; and

the R³ groups do not comprise exactly one methyl and exactly one -halo or the R³ groups do not comprise exactly two methyl groups at the 6 and 7 positions of the indoline ring when n is 0 and R² and every R⁹ are —H.

In one embodiment, three occurrences of Y are independently —C(R³)— and one occurrence of Y is —N—.

In another embodiment, two occurrences of Y are independently —C(R³)— and two occurrences of Y are —N—.

In yet another embodiment, one occurrence of Y is —C(R³)— and three occurrences of Y¹ are —N—.

In one embodiment, each occurrence of Y is —N—.

In one embodiment, each occurrence of Y is independently —C(R³)—.

In a specific embodiment, each occurrence of Y is —CH—.

In one embodiment, each occurrence of —C(R³)— is —CH—.

In one embodiment, X⁴ is —(CH₂)ₘ—C(O)O—.

In another embodiment, X⁴ is —(CH₂)ₘ—C(O)NH—.

In still another embodiment, X⁴ is —CH₂—.

In a further embodiment, X⁴ is —(CH₂)ₘ—SO₂—.

In another embodiment, X⁴ is —(CH₂)ₘ—C(O)NR²—.

In yet another embodiment, X⁴ is —C(O)—.

In one embodiment, n is 1.

In another embodiment, n is 0.

In one embodiment, m is 1.

In another embodiment, m is 0.

In one embodiment, each occurrence of R⁹ is —H.

In another embodiment, four occurrences of R⁹ are —H.

In still another embodiment, three occurrences of R⁹ are —H.

In one embodiment, one occurrence of R⁹ is C₁-C₆ alkyl.

In another embodiment, two occurrence of R⁹ are C₁-C₆ alkyl.

In still another embodiment, one occurrence of R⁹ is methyl.

In yet another embodiment, one occurrence of R⁹ is isopropyl.

In another embodiment, one occurrence of R⁹ is tert-butyl.

In one embodiment, R⁹ is -halo.

In another embodiment, at least one occurrence of R⁹ is —F.

In one embodiment, at least one occurrence of R⁹ is —O—C₁-C₆ alkyl.

In a specific embodiment, at least one occurrence of R⁹ is —O-methyl.

In one embodiment, R⁹ is in the para position on the phenyl ring to which it is attached.

In another embodiment, R⁹ is in an ortho position on the phenyl ring to which it is attached.

In still another embodiment, R⁹ is in a meta position on the phenyl ring to which it is attached.

In one embodiment, R² is H.

In another embodiment, R² is not H.

In yet another embodiment, R² is —C₁-C₆ alkyl.

In a specific embodiment, R² is methyl.

In another specific embodiment, R² is ethyl.

In yet another specific embodiment, R² is propyl.

In still another specific embodiment, R² is isopropyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —(CH₂)ₘC(O)O—(C₁-C₆ alkyl).

In another embodiment, R² is C₂-C₆ alkenyl.

In one embodiment, R² is allyl.

In another embodiment, R² is C₂-C₆ alkynyl.

In a specific embodiment, R² is —CH₂—C≡CH.

In another embodiment, R² is aryl.

In a specific embodiment, R² is phenyl.

In another embodiment, R² is substituted aryl.

In a yet another embodiment, R² is -phenyl substituted with at least one C₁-C₆ alkyl.

In a specific embodiment, R² is -phenyl substituted with at least one methyl.

In another embodiment, R² is -phenyl substituted with at least one -halo.

In another embodiment, R² is -phenyl substituted with at least one —F or —Cl.

In a specific embodiment, R² is -phenyl substituted with at least one —F or —Cl.

In another embodiment, R² is —(C₁-C₆ alkyl)-aryl.

In a specific embodiment, R² is -benzyl.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In an embodiment, R³ is —H.

In a specific embodiment, at least one occurrence of R³ is —CF₃.

In another specific embodiment, at least one occurrence of R³ is —OCF₃.

In one embodiment, at least one occurrence of R³ is —O—C₁-C₆ alkyl.

In still another specific embodiment, at least one occurrence of R³ is —O-methyl.

In one specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, at least one occurrence of R³ is -halo.

In a specific embodiment, at least one occurrence of R³ is —F or —Cl.

In another specific embodiment, two occurrences of R³ are fluorides.

In another specific embodiment, two occurrences of R³ are chlorides.

In one embodiment, a compound of Formula (VI) is in isolated and purified form.

Illustrative examples of Thiadiazole Compounds of Formula (VI) are set forth below:

| Compound | Name |
|---|---|
| 31 | 5′-phenyl-5-(trifluoromethoxy)-3′H-spiro[indole-3,2′-[1,3,4]thiadiazol]- |
| | 2(1H)-one |
| 32 | 5-methyl-1-(2-methylphenyl)-5′-(4-methylphenyl)-3′H-spiro[indole-3,2′- |
| | [1,3,4]thiadiazol]-2(1H)-one |
| 33 | 1-benzyl-5-methyl-5′-(4-methylphenyl)-3′H-spiro[indole-3,2′- |
| | [1,3,4]thiadiazol]-2(1H)-one |
| 34 | 5,7-dimethyl-5′-phenyl-3′H-spiro[indole-3,2′-[1,3,4]thiadiazol]-2(1H)-one |
| 35 | 5-methoxy-5′-phenyl-3′H-spiro[indole-3,2′-[1,3,4]thiadiazol]-2(1H)-one |
| 36 | 1-(4-chlorobenzoyl)-5′-(4-methoxyphenyl)-3′H-spiro[indole-3,2′- |
| | [1,3,4]thiadiazol]-2(1H)-one |
| 37 | 5′-(4-methylphenyl)-6-propyl-3′H-spiro[indole-3,2′-[1,3,4]thiadiazol]- |
| | 2(1H)-one |
| 38 | 1,5′-diphenyl-3′H-spiro[indole-3,2′-[1,3,4]thiadiazol]-2(1H)-one |
| 39 | 5,6-difluoro-5′-phenyl-3′H-spiro[indole-3,2′-[1,3,4]thiadiazol]-2(1H)-one |
| 40 | 4,7-dichloro-5′-phenyl-3′H-spiro[indole-3,2′-[1,3,4]thiadiazol]-2(1H)-one |
| 41 | 1-allyl-5′-phenyl-3′H-spiro[indole-3,2′-[1,3,4]thiadiazol]-2(1H)-one |
| 42 | 1-isopropyl-5′-phenyl-3′H-spiro[indole-3,2′-[1,3,4]thiadiazol]-2(1H)-one |
| 43 | 5-ethyl-5′-(4-fluorophenyl)-3′H-spiro[indole-3,2′-[1,3,4]thiadiazol]- |
| | 2(1H)-one |
| 44 | 5′-(4-tert-butylphenyl)-5-ethyl-3′H-spiro[indole-3,2′-[1,3,4]thiadiazol]- |
| | 2(1H)-one |
| 45 | 1-butyryl-5-ethyl-5′-(4-methylphenyl)-3′H-spiro[indole-3,2′- |
| | [1,3,4]thiadiazol]-2(1H)-one |
| 46 | 1-butyryl-5-ethyl-5′-(4-methoxyphenyl)-3′H-spiro[indole-3,2′- |
| | [1,3,4]thiadiazol]-2(1H)-one |

and pharmaceutically acceptable salts or hydrates thereof.

### 4.2.7 The Thiadiazole Compounds of Formula (VII)

In one embodiment, the invention provides compounds of the Formula (VII):

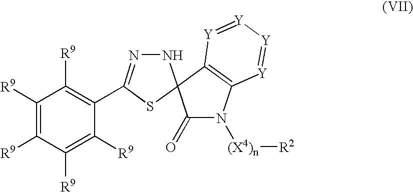

or a pharmaceutically acceptable salt or hydrate thereof, wherein X⁴, Y, n, R⁹ and R² are as defined above for the Thiadiazole Compounds of Formula (VII).

In one embodiment, three occurrences of Y are independently —C(R³)— and one occurrence of Y is —N—.

In another embodiment, two occurrences of Y are independently —C(R³)— and two occurrences of Y are —N—.

In yet another embodiment, one occurrence of Y is —C(R³)— and three occurrences of Y are —N—.

In one embodiment, each occurrence of Y is —N—.

In one embodiment, each occurrence of Y is independently —C(R³)—.

In a specific embodiment, each occurrence of Y is —CH—.

In one embodiment, each occurrence of —C(R³)— is —CH—.

In one embodiment, X⁴ is —(CH₂)ₘ—C(O)O—.

In another embodiment, X⁴ is —(CH₂)ₘ—C(O)NH—.

In still another embodiment, X⁴ is —CH₂—.

In a further embodiment, X⁴ is —(CH₂)ₘ—SO₂—.

In another embodiment, X⁴ is —(CH₂)ₘ—C(O)NR²—.

In yet another embodiment, X⁴ is —C(O)—.

In one embodiment, n is 1.

In another embodiment, n is 0.

In one embodiment, m is 1.

In another embodiment, m is 0.

In one embodiment, each occurrence of R⁹ is —H.

In another embodiment, four occurrences of R⁹ are —H.

In still another embodiment, three occurrences of R⁹ are —H.

In one embodiment, one occurrence of R⁹ is C₁-C₆ alkyl.

In another embodiment, two occurrence of R⁹ are C₁-C₆ alkyl.

In still another embodiment, one occurrence of R⁹ is methyl.

In yet another embodiment, one occurrence of R⁹ is isopropyl.

In another embodiment, one occurrence of R⁹ is tert-butyl.

In one embodiment, R⁹ is -halo.

In another embodiment, one occurrence of R⁹ is —F.

In one embodiment, at least one occurrence of R⁹ is —O—C₁-C₆ alkyl.

In a specific embodiment, at least one occurrence of R⁹ is —O-methyl.

In one embodiment, R⁹ is in the para position on the phenyl ring to which it is attached.

In another embodiment, R⁹ is in an ortho position on the phenyl ring to which it is attached.

In still another embodiment, R⁹ is in a meta position on the phenyl ring to which it is attached.

In one embodiment, R² is H.

In another embodiment, R² is not H.

In yet another embodiment, R² is —C₁-C₆ alkyl.

In a specific embodiment, R² is methyl.

In another specific embodiment, R² is ethyl.

In yet another specific embodiment, R² is propyl.

In still another specific embodiment, R² is isopropyl.

In one embodiment, R² is —(C₁-C₆ alkyl)-N(C₁-C₆ alkyl)₂.

In a specific embodiment, R² is —(CH₂)₂N(CH₃)₂.

In one embodiment, R² is —(CH₂)ₘC(O)O—(C₁-C₆ alkyl).

In another embodiment, R² is —C₂-C₆ alkenyl.

In one embodiment, R² is allyl.

In another embodiment, R² is —C₂-C₆ alkynyl.

In a specific embodiment, R² is —CH₂—C≡CH.

In another embodiment, R² is -aryl.

In a specific embodiment, R² is -phenyl.

In another embodiment, R² is substituted -aryl.

In a yet another embodiment, R² is -phenyl substituted with at least one C₁-C₆ alkyl.

In a specific embodiment, R² is -phenyl substituted with at least one methyl.

In a specific embodiment, R² is -phenyl substituted with —CF₃.

In a specific embodiment, R² is -phenyl substituted with —OCF₃.

In another embodiment, R² is -phenyl substituted with at least one -halo.

In a specific embodiment, R² is -phenyl substituted with at least one —F or —Cl.

In another specific embodiment, R² is -phenyl substituted with two fluorides or two chlorides.

In another embodiment, R² is —(C₁-C₆ alkyl)-aryl.

In a specific embodiment, R² is -benzyl.

In a specific embodiment, R² is -benzyl substituted with at least one C₁-C₆ alkyl.

In a specific embodiment, R² is -benzyl substituted with at least one -halo.

In a specific embodiment, R² is -benzyl substituted with at least one —CF₃.

In a specific embodiment, R² is -benzyl substituted with at least one —OCF₃.

In another embodiment, R² is —(C₁-C₆ alkyl)-(5 or 6-membered aromatic or non-aromatic heterocycle).

In another embodiment, R² is -5 or 6-membered aromatic or non-aromatic heterocycle.

In one embodiment, R³ is —H.

In one embodiment, at least one occurrence of R³ is —C₁-C₆ alkyl.

In a specific embodiment, at least one occurrence of R³ is methyl.

In another specific embodiment, at least one occurrence of R³ is ethyl.

In yet another specific embodiment, at least one occurrence of R³ is n-propyl.

In another specific embodiment, at least one occurrence of R³ is isopropyl.

In an embodiment, R³ is —H.

In a specific embodiment, at least one occurrence of R³ is —CF₃.

In another specific embodiment, at least one occurrence of R³ is —OCF₃.

In yet another specific embodiment, at least one occurrence of R³ is —O—C₁-C₆ alkyl.

In still another specific embodiment, at least one occurrence of R³ is —O-methyl.

In one specific embodiment, at least one occurrence of R³ is —NO₂.

In one embodiment, at least one occurrence of R³ is -5 or 6-membered aromatic or non-aromatic heterocycle.

In a specific embodiment, at least one occurrence of R³ is piperazin-1-yl.

In one embodiment, at least one occurrence of R³ is —(C₁-C₆ alkyl)-(5 or 6-membered aromatic or non-aromatic heterocycle).

In one embodiment, at least one occurrence of R³ is -(5 or 6-membered aromatic or non-aromatic heterocycle)-aryl.

In one embodiment, at least one occurrence of R³ is -(5 or 6-membered aromatic or non-aromatic heterocycle)-CH₂-aryl.

In one embodiment, two occurrences of R³ are independently C₁-C₆ alkyl.

In a specific embodiment, two occurrences of R³ are methyl.

In one embodiment, at least one occurrence of R³ is -halo.

In a specific embodiment, two occurrences of R³ are -halo.

In a specific embodiment, at least one occurrence of R³ is —Br.

In a specific embodiment, at least one occurrence of R³ is —F.

In a specific embodiment, at least one occurrence of R³ is —Cl.

In one embodiment, a compound of Formula (VII) is in isolated and purified form.

Illustrative examples of Thiadiazole Compounds of Formula (VII) are set forth below:

| Compound | Name |
|---|---|
| 47 | 5-ethyl-5′-phenyl-3′H-spiro[indole-3,2′-[1,3,4]thiadiazol]-2(1H)-one |
| 48 | 5-ethyl-5′-(4-methylphenyl)-3′H-spiro[indole-3,2′-[1,3,4]thiadiazol]- |
| | 2(1H)-one |
| 49 | 5-methyl-5′-(4-methylphenyl)-3′H-spiro[indole-3,2′-[1,3,4]thiadiazol]- |
| | 2(1H)-one |
| 50 | 5′-(4-methylphenyl)-3′H-spiro[indole-3,2′-[1,3,4]thiadiazol]-2(1H)-one |
| 51 | 1-methyl-5′-(4-methylphenyl)-3′H-spiro[indole-3,2′-[1,3,4]thiadiazol]- |
| | 2(1H)-one |
| 52 | 1-methyl-5′-phenyl-3′H-spiro[indoline-3,2′-[1,3,4]thiadiazol]-2-one |
| 53 | 5′-phenyl-3′H-spiro[indoline-3,2′-[1,3,4]thiadiazol]-2-one |
| 54 | 5-bromo-5′-phenyl-3′H-spiro[indoline-3,2′-[1,3,4]thiadiazol]-2-one |
| 55 | 5-methyl-5′-phenyl-3′H-spiro[indoline-3,2′-[1,3,4]thiadiazol]-2-one |
| 56 | 1-acetyl-5′-phenyl-3′H-spiro[indoline-3,2′-[1,3,4]thiadiazol]-2-one |

and pharmaceutically acceptable salts or hydrates thereof.

The Thiadiazole Compounds of the invention may contain one or more asymmetric centers, and can thus give rise to optical isomers and diastereomers. While depicted without respect to stereochemistry in the compounds or pharmaceutically acceptable salts of compounds of the present invention, the present invention includes such optical isomers and diastereomers, as well as racemic and resolved, enantiomerically pure R and S stereoisomers, and also other mixtures of the R and S stereoisomers and pharmaceutically acceptable salts thereof. Where a stereoisomer is provided, it can in some embodiments be provided substantially free of its corresponding opposite enantiomer.

In addition, the compounds and pharmaceutically acceptable salts of compounds of the present invention may exist as tautomers. Such tautomers can be transient or isolatable as a stable product. These tautomers are within the scope of the present invention.

### 4.3 Methods for Making the Thiadiazole Compounds

Examples of methods that are useful for making Dibenzonaphthyridine Derivatives are set forth in the Examples below and generalized in Schemes 1-4.

Scheme 1 sets forth a method for making Thiadiazole Compounds of Formulas (I)-(VII):

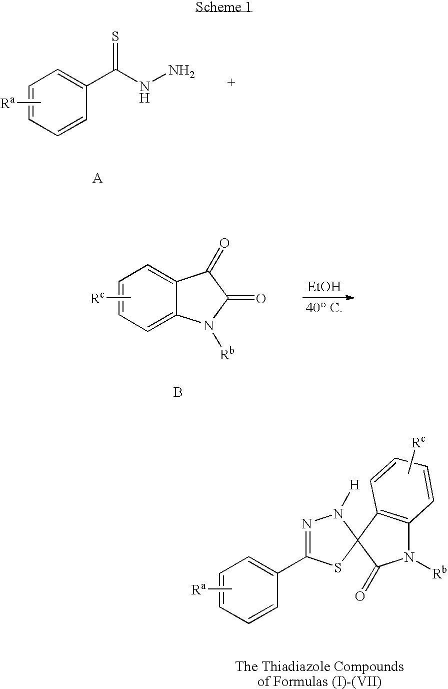

where R^{a} is R¹, R⁴, R⁶, or R⁹; R^{b} is R² or R⁷; and R^{c} is R³ or R⁸, as defined above for the compounds of Formulae (I)-(VII).

A thiobenzoylhydrazine compound of Formula A can be reacted with an isatin compound of Formula B under refluxing conditions in ethanol according to the procedure set forth in Tomchin, Zhurnal Organicheskoi Khimii. 17: 1561 (1981) and Tomchin, Zhurnal Organicheskoi Khimii. 23:1305 (1987).

Scheme 2 sets forth a method for making the thiobenzoylhydrazine compounds of Formula A.

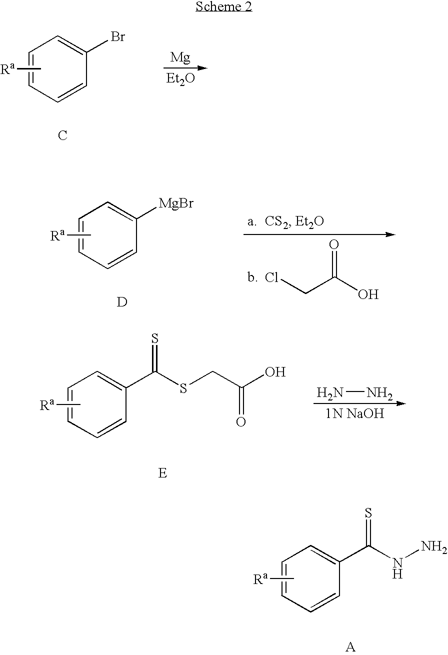

An aryl magnesium bromide of Formula D can be reacted with carbon disulfide in ether and subsequently alkylated using chloroacetic acid as set forth in Werbel et. al., J. Heterocyclic Chem. 1979, 16, 881, to provide a carboxylic acid of Formula E, which can then reacted with hydrazine under basic conditions according to the procedure set forth in Reeve et. al., Can. J. Chem. 57:444 (1979) to provide a thiobenzoylhydrazine compound of Formula A. The aryl magnesium bromides of Formula D can be purchased commercially or prepared from commercially available aryl bromides of Formula C, using methods disclosed in Kovalainen et al., J. Med. Chem. 1999, 42, 1193.

Scheme 3 sets forth a method for making the isatin compounds of Formula B, wherein R² is other than hydrogen.

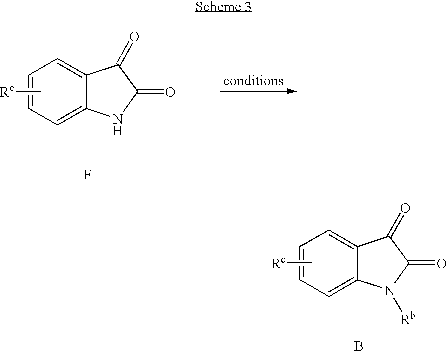

Various isatin compounds of Formula B can be purchased commercially or prepared from isatin intermediates of Formula F. Isatin derivatives of Formula F can be prepared using the methods set forth in Ma et al., Tetrahedron Letters, 41:9089 (2000); Rossiter, Tetrahedron Letters, 43:4671 (2002); and Rewcastle et al., Journal of Medicinal Chemistry, 34:217 (1991). Isatin derivatives of Formula B can be synthesized using methods set forth in Makhija, et al., Bioorganic & Medicinal Chemistry, 12:2317 (2004); Chiyanzu, et al., Bioorganic & Medicinal Chemistry Letters, 13:3527 (2003); Azizian, et al., Synthetic Communications, 33:789 (2003); Singh, et al., Journal of Organic Chemistry, 66:6263 (2001); and Lam, et al., Tetrahedron Letters, 42:3415 (2001).

Scheme 4 sets forth a method for making the isatin intermediates of Formula F.

Scheme 4 sets forth a method for making the isatin compounds of Formula F which are useful intermediates for making the isatin compounds of Formula B, where R² is other than hydrogen.

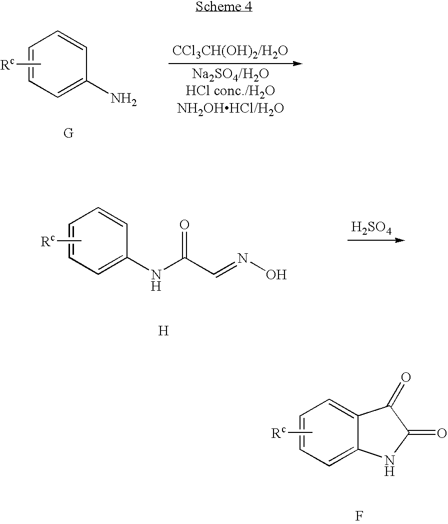

To an aqueous solution of chloral hydrate is sequentially added an aqueous solution of Na₂SO₄, an aqueous solution of a phenylamine of Formula G, and concentrated hydrochloric acid. To the resultant mixture is added an aqueous solution of hydroxylamine hydrochloride at elevated temperature to provide a hydroxyimino intermediate of Formula H, which is then treated with concentrated H₂SO₄ to provide an isatin intermediate of Formula F. The conversion of an intermediate of Formula F to a compound of Formula B can be accomplished by methods well known to one of skill in the art.

### 4.4 Therapeutic/Prophylactic Administration and Compositions of the Invention

Due to their activity, the Thiadiazole Compounds are advantageously useful in human and veterinary medicine. As described above, the Thiadiazole Compounds are useful for treating or preventing a Condition in a mammal in need thereof.

When administered to a mammal, the Thiadiazole Compounds can be administered as a component of a composition that comprises a physiologically acceptable carrier or vehicle. The present compositions, which comprise a Thiadiazole Compound, can be administered orally. The Thiadiazole Compounds can also be administered by any other convenient route, for example, by infusion or bolus injection, by absorption through epithelial or mucocutaneous linings (e.g., oral, rectal, or intestinal mucosa), by intratracheal administration, or by inhalation, and can be administered together with another biologically active agent. Administration can be systemic or local. Various known delivery systems, including encapsulation in liposomes, microparticles, microcapsules, and capsules, can be used.

Methods of administration include, but are not limited to, intradermal, intratracheal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration results in the release of the Thiadiazole Compounds into the bloodstream. The mode of administration can be left to the discretion of the practitioner.

In one embodiment, the Thiadiazole Compounds are administered orally.

In another embodiment, the Thiadiazole Compounds are administered intravenously.

In another embodiment, the Thiadiazole Compounds are administered topically.

In still another embodiment, the Thiadiazole Compounds are administered via inhalation.

In other embodiments, it can be desirable to administer the Thiadiazole Compounds locally. This can be achieved, for example, and not by way of limitation, by local infusion during surgery, topical application, e.g., in conjunction with a wound dressing after surgery, by injection, by means of a catheter, by intubation, by means of a suppository or enema, or by means of an implant, said implant being of a porous, non-porous, or gelatinous material, including membranes, such as sialastic membranes, or fibers.

In certain embodiments, it can be desirable to introduce the Thiadiazole Compounds into the central nervous system, circulatory system or gastrointestinal tract by any suitable route, including intraventricular, intrathecal injection, paraspinal injection, epidural injection, enema, and by injection adjacent to a peripheral nerve. Intraventricular injection can be facilitated by an intraventricular catheter, for example, attached to a reservoir, such as an Ommaya reservoir.

Pulmonary administration can also be employed, e.g., by use of an inhaler of nebulizer, by intubation, and formulation with an aerosolizing agent, or via perfusion in a fluorocarbon or synthetic pulmonary surfactant. In certain embodiments, the Thiadiazole Compounds can be formulated as a suppository, with traditional binders and excipients such as triglycerides.

In another embodiment the Thiadiazole Compounds can be delivered in a vesicle, in particular a liposome (see Langer, Science 249:1527-1533 (1990) and Lopez-Berestein et al., Liposomes in the Therapy of Infectious Disease and Cancer, 317-327 and 353-365 (1989)).

In yet another embodiment the Thiadiazole Compounds can be delivered in a controlled-release system or sustained-release system (see, e.g., Goodson, in Medical Applications of Controlled Release, supra, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed. Eng. 14:201 (1987); Buchwald et al., Surgery 88:507 (1980); and Saudek et al., N. Engl. J. Med. 321:574 (1989)). In another embodiment polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment a controlled- or sustained-release system can be placed in proximity of a target of the Thiadiazole Compounds, e.g., the spinal column, brain, colon, skin, heart, lung, trachea or gastrointestinal tract, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment the physiologically acceptable excipients are sterile when administered to a mammal. Water can be a particularly useful excipient when the Thiadiazole Compound is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, tablets, pills, pellets, capsules, capsules containing liquids, powders, sustained-release formulations, suppositories, emulsions, aerosols, sprays or any other form suitable for use. In one embodiment the composition is in the form of a capsule. Other examples of suitable physiologically acceptable excipients are described in Remington's Pharmaceutical Sciences 1447-1676 (Alfonso R. Gennaro eds., 19th ed. 1995), incorporated herein by reference.

In one embodiment the Thiadiazole Compounds are formulated in accordance with routine procedures as a composition adapted for oral administration to human beings. Compositions for oral delivery can be in the form of tablets, lozenges, aqueous or oily suspensions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active platform driving a Thiadiazole Compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment the excipients are of pharmaceutical grade.

In another embodiment the Thiadiazole Compounds can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. The compositions' components can be supplied either separately or mixed together in unit dosage form, for example, as a dry lyophilized powder or water-free concentrate in a hermetically sealed container such as an ampoule or sachette indicating the quantity of Thiadiazole Compound. Where the Thiadiazole Compounds are to be administered by infusion, they can be dispensed, for example, with an infusion bottle containing sterile pharmaceutical grade water or saline. Where the Thiadiazole Compounds are administered by injection, an ampoule of sterile water for injection or saline can be provided so that the ingredients can be mixed prior to administration.

The Thiadiazole Compounds can be administered by controlled-release or sustained-release means or by delivery devices that are well known to those skilled in the art. Such dosage forms can be used to provide controlled- or sustained-release of one or more active ingredients using, for example, hydroxypropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release.

In one embodiment a controlled- or sustained-release composition comprises a minimal amount of a Thiadiazole Compound to treat or prevent the Condition in a minimal amount of time. Advantages of controlled- or sustained-release compositions include extended activity of the drug, reduced dosage frequency, and increased patient compliance. In addition, controlled- or sustained-release compositions can favorably affect the time of onset of action or other characteristics, such as blood levels of the Thiadiazole Compound, and can thus reduce the occurrence of adverse side effects.

Controlled- or sustained-release compositions can initially release an amount of a Thiadiazole Compound that promptly produces the desired therapeutic or prophylactic effect, and gradually and continually release other amounts of the Thiadiazole Compound to maintain this level of therapeutic or prophylactic effect over an extended period of time. To maintain a constant level of the Thiadiazole Compound in the body, the Thiadiazole Compound can be released from the dosage form at a rate that will replace the amount of Thiadiazole Compound being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

The amount of the Thiadiazole Compound that is effective for treating or preventing a Condition can be determined by standard clinical techniques. In addition, in vitro or in vivo assays can optionally be employed to help identify optimal dosage ranges. The precise dose to be employed can also depend on the route of administration, and the seriousness of the condition being treated and can be decided according to the judgment of a health-care practitioner. Suitable effective dosage amounts, however, range from about 10 micrograms to about 5 grams about every 4 h, although they are typically about 500 mg or less per every 4 hours. In one embodiment the effective dosage is about 0.01 mg, 0.5 mg, about 1 mg, about 50 mg, about 100 mg, about 200 mg, about 300 mg, about 400 mg, about 500 mg, about 600 mg, about 700 mg, about 800 mg, about 900 mg, about 1 g, about 1.2 g, about 1.4 g, about 1.6 g, about 1.8 g, about 2.0 g, about 2.2 g, about 2.4 g, about 2.6 g, about 2.8 g, about 3.0 g, about 3.2 g, about 3.4 g, about 3.6 g, about 3.8 g, about 4.0 g, about 4.2 g, about 4.4 g, about 4.6 g, about 4.8 g, or about 5.0 g, every 4 hours. Equivalent dosages can be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be determined according to the judgment of a health-care practitioner. The effective dosage amounts described herein refer to total amounts administered; that is, if more than one Thiadiazole Compound is administered, the effective dosage amounts correspond to the total amount administered.

The amount of a Thiadiazole Compound that is effective for treating or preventing a Condition typically range from about 0.01 mg/kg to about 100 mg/kg of body weight per day, in one embodiment, from about 0.1 mg/kg to about 50 mg/kg body weight per day, and in another embodiment, from about 1 mg/kg to about 20 mg/kg of body weight per day.

When a Thiadiazole Compound is a component of a solution that is useful for maintaining the viability of an organ ex vivo, the concentration of the Thiadiazole Compound in the solution that is effective for maintaining the viability of the organ is between about 1 nM to about 1 mM.

The Thiadiazole Compounds can be assayed in vitro or in vivo for the desired therapeutic or prophylactic activity prior to use in humans. Animal model systems can be used to demonstrate safety and efficacy.

The present methods for treating or preventing a Condition can further comprise administering another therapeutic agent to the mammal being administered a Thiadiazole Compound. In one embodiment the other therapeutic agent is administered in an effective dose.

Effective doses of the other therapeutic agents are well known to those skilled in the art. However, it is well within the skilled artisan's purview to determine the other therapeutic agent's optimal effective dose range. In one embodiment of the invention, where, another therapeutic agent is administered to a mammal, the effective dose of the Thiadiazole Compound is less than its effective dose would be where the other therapeutic agent is not administered. In this case, without being bound by theory, it is believed that the Thiadiazole Compounds and the other therapeutic agent act synergistically.

In one embodiment the other therapeutic agent is an anti-inflammatory agent. Examples of useful anti-inflammatory agents include, but are not limited to, adrenocorticosteroids, such as cortisol, cortisone, fluorocortisone, prednisone, prednisolone, 6α-methylprednisolone, triamcinolone, betamethasone, and dexamethasone; and non-steroidal anti-inflammatory agents (NSAIDs), such as aspirin, acetaminophen, indomethacin, sulindac, tolmetin, diclofenac, ketorolac, ibuprofen, naproxen, flurbiprofen, ketoprofen, fenoprofen, oxaprozin, mefenamic acid, meclofenamic acid, piroxicam, meloxicam, nabumetone, rofecoxib, celecoxib, etodolac, and nimesulide.

In a further embodiment the other therapeutic agent is an anti-cardiovascular-disease agent. Examples of useful anti-cardiovascular-disease agents include, but are not limited to, carnitine; thiamine; lidocaine; amiodarone; procainamide; mexiletine; bretylium tosylate; propanolol; sotalol; and muscarinic receptor antagonists, such as atropine, scopolamine, homatropine, tropicamide, pirenzipine, ipratropium, tiotropium, and tolterodine.

A Thiadiazole Compound and the other therapeutic agent can act additively or, in one embodiment, synergistically. In one embodiment, a Thiadiazole Compound is administered concurrently with another therapeutic agent. In another embodiment, the present compositions can further comprise another therapeutic agent. In a further embodiment, a composition comprising an effective dose of a Thiadiazole Compound and an effective dose of another therapeutic agent can be administered. Alternatively, a composition comprising an effective dose of a Thiadiazole Compound and a different composition comprising an effective dose of another therapeutic agent can be concurrently administered. In another embodiment, an effective dose of a Thiadiazole Compound is administered prior or subsequent to administration of an effective dose of another therapeutic agent. In this embodiment, the Thiadiazole Compound is administered while the other therapeutic agent exerts its therapeutic effect, or the other therapeutic agent is administered while the Thiadiazole Compound exerts its preventative or therapeutic effect for treating or preventing a Condition.

A composition of the invention can be prepared using a method comprising admixing a Thiadiazole Compound and a physiologically acceptable carrier or excipient. Admixing can be accomplished using methods well known for admixing a compound (or salt) and a physiologically acceptable carrier or excipient.

### 4.5 Therapeutic or Prophylactic Uses of the Thiadiazole Compounds

In one embodiment, the Thiadiazole Compounds of the present invention and compositions thereof are useful as metalloproteinase modulators.

In another embodiment, the Thiadiazole Compounds of the present invention and compositions thereof are useful for treating or preventing a condition.

In one embodiment, the invention provides a method for treating or preventing a condition, such as a metalloproteinase-related disorder, comprising administering to a mammal in need thereof an effective dose of a Thiadiazole Compound or a composition thereof.

The Thiadiazole Compounds and compositions thereof are useful for treating or preventing the following disorders: an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

In one embodiment, the disorder is osteoarthritis.

In one embodiment, the present invention provides a method of treating or preventing a disorder in a mammal in need thereof, which comprises administering an effective dose of a Thiadiazole Compound or a pharmaceutically acceptable salt or hydrate thereof, wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

### 4.6 Kits

The invention encompasses kits that can simplify the administration of the Thiadiazole Compounds or compositions of the invention to a mammal.

A typical kit of the invention comprises a unit dosage of a Thiadiazole Compound. In one embodiment, the unit dosage form is in a container, which can be sterile, containing an effective dose of a Thiadiazole Compound, i.e. a pharmaceutical composition comprising a Thiadiazole Compound, and a physiologically acceptable vehicle. In another embodiment, the unit dosage form is in a container containing an effective dose of a Thiadiazole Compound, i.e. a pharmaceutical composition comprising a Thiadiazole Compound, as a lyophilizate or pharmaceutically acceptable salt. In this instance, the kit can further comprise another container that contains a solution useful for the reconstitution of the lyophilizate or dissolution of the salt. The kit can also comprise a label or printed instructions for use of the Thiadiazole Compounds.

In a further embodiment, the kit comprises a unit dosage form of a composition of the invention.

Kits of the invention can further comprise one or more devices that are useful for administering the unit dosage forms of the Thiadiazole Compounds or a composition of the invention. Examples of such devices include, but are not limited to, a syringe, a drip bag, a patch or an enema, which optionally contain the unit dosage forms.

The present invention is not to be limited in scope by the specific embodiments disclosed in the examples which are intended as illustrations of a few aspects of the invention and any embodiments that are functionally equivalent are within the scope of this invention.

### 5. EXAMPLES

### Example 1

### General Method for Making the Thiadiazole Compounds

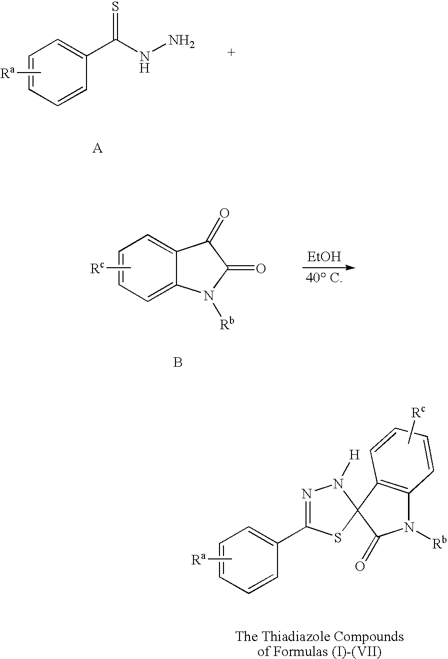

A thiobenzoylhydrazine compound of Formula A (1 eq) is diluted with ethanol to provide a solution having a concentration of about 0.2 mmol/mL and to the resultant solution is added an isatin analog of Formula B (1.2 eq). The resulting reaction is heated to 40° C. and allowed to stir at this temperature for about 2 hours. The reaction mixture is allowed to cool to room temperature to provide a suspension, which is filtered and the collected solid crude product is used as is or further purified via recrystallization from an appropriate solvent or purified using preparative HPLC. If no suspension is formed upon cooling the reaction mixture, water is added to the reaction mixture until the product oils out. In this case, the liquid is separated from the oil to provide the illustrative Thiadiazole Compound further which can be further purified via recrystallization or preparative HPLC.

### Preparative HPLC Method

If purification via preparative HPLC is desired, the following conditions can be used to purify the Thiadiazole Compounds. The crude residue is dissolved in DMSO:MeCN (3:1), filtered through a 0.45 μm GMF, and purified on a Gilson HPLC, using a Phenomenex LUNA C₁₈ column: 60 mm×21.20 mm I.D., 5 um particle size: with ACN/water (containing 0.2% TFA or Et₃N) gradient elution. The appropriate fractions were analyzed by LC/MS as described below. Combining pure fractions and evaporating the solvent in a Speed-Vac isolated the title compound.

### Example 1A

### Preparation of 1-Butyryl-5-ethylisatin (K)

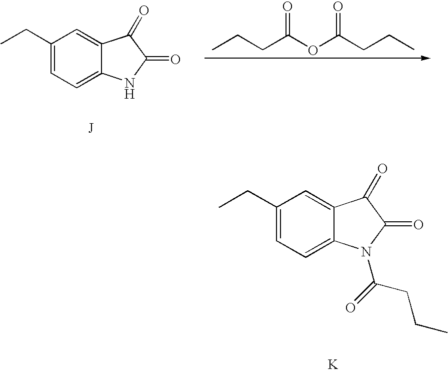

5-ethylisatin (1 g, 5.7 mmol) (J) was dissolved in 3 ml of butyric anhydride and the solution was refluxed for 48 h. The reaction was cooled to room temperature and the precipitate was filtered and dried to give 1-Butyryl-5-ethylisatin (K), which is a compound of Formula B where R² is not hydrogen. Yield: 75%.

### Example 1B

### Preparation of 5-ethylisatin (J)

To a solution of chloral hydrate (15 g, 90.75 mmol) in H₂O (86 ml) were added dropwise an aqueous solution of Na₂SO₄ (80 g in 150 ml), a solution of 4-ethylaniline (10 g, 82.5 mmol) in H₂O (32 ml) and HCl 36% (7.5 ml), and a solution of hydroxylamine hydrochloride (18 g, 69.49 mmol) in H₂O (70 ml). The resultant mixture was refluxed for 2 h, during which formation of a precipitate was observed, then the mixture was stirred overnight at room temperature. The precipitate was filtered, dried under vacuum and crystallized from ethanol/water to obtain N-(4-Ethyl-phenyl)-2-[(E)-hydroxyimino]-acetamide. Yield: 75%.

Next, 92 mL of concentrated H₂SO₄ were pre-heated to 50° C. under magnetic stirring. N-(4-Ethyl-phenyl)-2-[(E)-hydroxyimino]-acetamide (11.9 g, 61.9 mmol) was added portionwise keeping the temperature below 65° C. The mixture was heated at 60° C. for 1 hour, then cooled at room temperature and quenched with ice (300 mL). The aqueous phase was extracted with ethyl acetate, washed with NaHCO₃ and dried over Na₂SO₄. After evaporation of the solvent, the brown crude residue was purified by silica gel chromatography, eluting with a mixture of hexanes and ethylacetate. 8 grams of 5-ethylisatin (J) as a bright-orange solid were isolated. Yield: 74%.

### Example 2

### Preparation of 5′-(4-methoxyphenyl)-5,7-dimethyl-3′H-spiro[indole3,2′-[1,3,4]thiadiazol]-2(1H)-one (Compound 1)

Using the method set forth in Example 1 and using 4-methoxybenzothiohydrazide as the thiobenzoylhydrazine compound and 5,7-dimethylindoline-2,3-dione as the isatin compound, Compound 1 was prepared.

### Example 3

### Preparation of 5′-(4-methoxyphenyl)-1-methyl-3′H-spiro[indole-3,2′-[1,3,4]thiadiazol]-2(1H)-one (Compound 2)

Using the method set forth in Example 1 and using 4-methoxybenzothiohydrazide as the thiobenzoylhydrazine compound and 1-methylindoline-2,3-dione as the isatin compound, Compound 2 was prepared.

### Example 4

### Preparation of 5′-(4-methoxyphenyl)-3′H-spiro[indole-3,2′-[1,3,4]thiadiazol]-2(1H)-one (Compound 3)

Using the method set forth in Example 1 and using 4-methoxybenzothiohydrazide as the thiobenzoylhydrazine compound and indoline-2,3-dione as the isatin compound, Compound 3 was prepared.

### Example 5

### Preparation of 5-bromo-5′-(4-methoxyphenyl)-3′H-spiro[indole-3,2′-[1,3,4]thiadiazol]-2(1H)-one (Compound 4)

Using the method set forth in Example 1 and using 4-methoxybenzothiohydrazide as the thiobenzoylhydrazine compound and 5-bromoindoline-2,3-dione as the isatin compound, Compound 4 was prepared.

### Example 6

### Preparation of 5′-(4-methoxyphenyl)-5-methyl-3′H-spiro[indole-3,2′-[1,3,4]thiadiazol]-2(1H)-one (Compound 5)

Using the method set forth in Example 1 and using 4-methoxybenzothiohydrazide as the thiobenzoylhydrazine compound and 5-methylindoline-2,3-dione as the isatin compound, Compound 5 was prepared.

### Example 7

### Preparation of 5-methoxy-5′-(4-methoxyphenyl)-3′H-spiro[indole-3,2′-[1,3,4]thiadiazol]-2(1H)-one (Compound 6)

Using the method set forth in Example 1 and using 4-methoxybenzothiohydrazide as the thiobenzoylhydrazine compound and 5-methoxyindoline-2,3-dione as the isatin compound, Compound 6 was prepared.

### Example 8

### Preparation of 5′-(4-methoxyphenyl)-1-prop-2-yn-1-yl-3′H-spiro[indole-3,2′-[1,3,4]thiadiazol]-2(1H)-one (Compound 7)

Using the method set forth in Example 1 and using 4-methoxybenzothiohydrazide as the thiobenzoylhydrazine compound and 1-(prop-2-ynyl)indoline-2,3-dione as the isatin compound, Compound 7 was prepared.

### Example 9

### Preparation of 5′-(4-methoxyphenyl)-1-[3-(trifluoromethyl)phenyl]-3′H-spiro[indole-3,2′-[1,3,4]thiadiazol]-2(1H)-one (Compound 8)

Using the method set forth in Example 1 and using 4-methoxybenzothiohydrazide as the thiobenzoylhydrazine compound and 1-(3-trifluoromethylphenyl)indoline-2,3-dione as the isatin compound, Compound 8 was prepared.

### Example 10

### Preparation of ethyl[5′-(4-methoxyphenyl)-2-oxo-3′H-spiro[indole-3,2′-[1,3,4]thiadiazol]-1(2H)-yl]acetate (Compound 9)

Using the method set forth in Example 1 and using 4-methoxybenzothiohydrazide as the thiobenzoylhydrazine compound and ethyl-2-(2,3-dioxoindolin-1-yl)acetate as the isatin compound, Compound 9 was prepared.

### Example 11

### Preparation of 1-benzyl-5′-(4-methoxyphenyl)-3′H-spiro[indole-3,2′-[1,3,4]thiadiazol]-2(1H)-one (Compound 10)

Using the method set forth in Example 1 and using 4-methoxybenzothiohydrazide as the thiobenzoylhydrazine compound and 1-benzylindoline-2,3-dione as the isatin compound, Compound 10 was prepared.

### Example 12

### Preparation of 1-[2-(dimethylamino)ethyl]-5′-(4-methoxyphenyl)-3′H-spiro[indole-3,2′-[1,3,4]thiadiazol]-2(1H)-one (Compound 11)

Using the method set forth in Example 1 and using 4-methoxybenzothiohydrazide as the thiobenzoylhydrazine compound and 1-(2-(dimethylamino)ethyl)indolin-2,3-dione as the isatin compound, Compound 11 was prepared.

### Example 13

### Preparation of 6-chloro-5′-(4-methoxyphenyl)-3′H-spiro[indole-3,2′-[1,3,4]thiadiazol]-2(1H)-one (Compound 12)

Using the method set forth in Example 1 and using 4-methoxybenzothiohydrazide as the thiobenzoylhydrazine compound and 6-chloroindoline-2,3-dione as the isatin compound, Compound 12 was prepared.

### Example 14

### Preparation of 5-chloro-5′-(4-methoxyphenyl)-3′H-spiro[indole-3,2′-[1,3,4]thiadiazol]-2(1H)-one (Compound 13)

Using the method set forth in Example 1 and using 4-methoxybenzothiohydrazide as the thiobenzoylhydrazine compound and 5-chloroindoline-2,3-dione as the isatin compound, Compound 13 was prepared.

### Example 15

### Preparation of 4-(4-benzylpiperazin-1-yl)-5′-(4-methoxyphenyl)-3′H-spiro[indole-3,2′-[1,3,4]thiadiazol]-2(1H)-one (Compound 14)

Using the method set forth in Example 1 and using 4-methoxybenzothiohydrazide as the thiobenzoylhydrazine compound and 4-(4-benzylpiperazin-1-yl)indoline-2,3-dione as the isatin compound, Compound 14 was prepared.

### Example 16

### Preparation of 5′-(4-methoxyphenyl)-6-propyl-3′H-spiro[indole-3,2′-[1,3,4]thiadiazol]-2(1H)-one (Compound 15)

Using the method set forth in Example 1 and using 4-methoxybenzothiohydrazide as the thiobenzoylhydrazine compound and 6-propylindoline-2,3-dione as the isatin compound, Compound 15 was prepared.

### Example 17

### Preparation of 5′-(4-methoxyphenyl)-5-nitro-3′H-spiro[indole-3,2′-[1,3,4]thiadiazol]-2(1H)-one (Compound 16)

Using the method set forth in Example 1 and using 4-methoxybenzothiohydrazide as the thiobenzoylhydrazine compound and 5-nitroindoline-2,3-dione as the isatin compound, Compound 16 was prepared.

### Example 18

### Preparation of 6-chloro-5′-(4-methoxyphenyl)-7-methyl-3′H-spiro[indole-3,2′-[1,3,4]thiadiazol]-2(1H)-one (Compound 17)

Using the method set forth in Example 1 and using 4-methoxybenzothiohydrazide as the thiobenzoylhydrazine compound and 6-chloro-7-methylindoline-2,3-dione as the isatin compound, Compound 17 was prepared.

### Example 19

### Preparation of 5′-(4-methoxyphenyl)-7-methyl-3′H-spiro[indole-3,2′-[1,3,4]thiadiazol]-2(1H)-one (Compound 18)

Using the method set forth in Example 1 and using 4-methoxybenzothiohydrazide as the thiobenzoylhydrazine compound and 7-methylindoline-2,3-dione as the isatin compound, Compound 18 was prepared.

### Example 20

### Preparation of 5′-(4-methoxyphenyl)-6-methyl-3′H-spiro[indole-3,2′-[1,3,4]thiadiazol]-2(1H)-one (Compound 19)

Using the method set forth in Example 1 and using 4-methoxybenzothiohydrazide as the thiobenzoylhydrazine compound and 6-methylindoline-2,3-dione as the isatin compound, Compound 19 was prepared.

### Example 21

### Preparation of 7-methoxy-5′-(4-methoxyphenyl)-6-methyl-3′H-spiro[indole-3,2′-[1,3,4]thiadiazol]-2(1H)-one (Compound 20)

Using the method set forth in Example 1 and using 4-methoxybenzothiohydrazide as the thiobenzoylhydrazine compound and 6-methyl-7-methyoxyindoline-2,3-dione as the isatin compound, Compound 20 was prepared.

### Example 22

### Preparation of 1-ethyl-5-fluoro-5′-(4-methoxyphenyl)-6-(4-methylpiperazin-1-yl)-3′H-spiro[indole-3,2′-[1,3,4]thiadiazol]-2(1H)-one (Compound 21)

Using the method set forth in Example 1 and using 4-methoxybenzothiohydrazide as the thiobenzoylhydrazine compound and 1-ethyl-5-fluoro-6-(4-methylpiperazin-1-yl)indoline-2,3-dione as the isatin compound, Compound 21 was prepared.

### Example 23

### Preparation of 1-benzyl-5′-(4-methoxyphenyl)-5-methyl-3′H-spiro[indole-3,2′-[1,3,4]thiadiazol]-2(1H)-one (Compound 22)

Using the method set forth in Example 1 and using 4-methoxybenzothiohydrazide as the thiobenzoylhydrazine compound and 1-benzyl-5-methylindoline-2,3-dione as the isatin compound, Compound 22 was prepared.

### Example 24

### Preparation of 5′-(4-methoxyphenyl)-5-methyl-1-(2-methylphenyl)-3′H-spiro[indole-3,2′-[1,3,4]thiadiazol]-2(1H)-one (Compound 23)

Using the method set forth in Example 1 and using 4-methoxybenzothiohydrazide as the thiobenzoylhydrazine compound and 1-(2-methylphenyl)-5-methylindoline-2,3-dione as the isatin compound, Compound 23 was prepared.

### Example 25

### Preparation of 5′-(4-methoxyphenyl)-5-methyl-1-(4-methylphenyl)-3′H-spiro[indole-3,2′-[1,3,4]thiadiazol]-2(1H)-one (Compound 24)

Using the method set forth in Example 1 and using 4-methoxybenzothiohydrazide as the thiobenzoylhydrazine compound and 1-(4-methylphenyl)-5-methylindoline-2,3-dione as the isatin compound, Compound 24 was prepared.

### Example 26

### Preparation of 1-(4-chlorophenyl)-5′-(4-methoxyphenyl)-5-methyl-3′H-spiro[indole-3,2′-[1,3,4]thiadiazol]-2(1H)-one (Compound 25)

Using the method set forth in Example 1 and using 4-methoxybenzothiohydrazide as the thiobenzoylhydrazine compound and 1-(4-chlorophenyl)-5-methylindoline-2,3-dione as the isatin compound, Compound 25 was prepared.

### Example 27

### Preparation of 1-(3,4-dichlorophenyl)-5′-(4-methoxyphenyl)-5-methyl-3′H-spiro[indole-3,2′-[1,3,4]thiadiazol]-2(1H)-one (Compound 26)

Using the method set forth in Example 1 and using 4-methoxybenzothiohydrazide as the thiobenzoylhydrazine compound and 1-(3,4-dichlorophenyl)-5-methylindoline-2,3-dione as the isatin compound, Compound 26 was prepared.

### Example 28

### Preparation of 5′-(4-methoxyphenyl)-5-methyl-1-(3-nitrophenyl)-3′H-spiro[indole-3,2′-[1,3,4]thiadiazol]-2(1H)-one (Compound 27)

Using the method set forth in Example 1 and using 4-methoxybenzothiohydrazide as the thiobenzoylhydrazine compound and 1-(3-nitrophenyl)-5-methylindoline-2,3-dione as the isatin compound, Compound 27 was prepared.

### Example 29

### Preparation of 1-benzyl-5′-(4-methoxyphenyl)-6-propyl-3′H-spiro[indole-3,2′-[1,3,4]thiadiazol]-2(1H)-one (Compound 28)

Using the method set forth in Example 1 and using 4-methoxybenzothiohydrazide as the thiobenzoylhydrazine compound and 1-benzyl-6-propylindoline-2,3-dione as the isatin compound, Compound 28 was prepared.

### Example 30

### Preparation of 5-ethyl-5′-(4-methoxyphenyl)-3′H-spiro[indole-3,2′-[1,3,4]thiadiazol]-2(1H)-one (Compound 29)

Using the method set forth in Example 1 and using 4-methoxybenzothiohydrazide as the thiobenzoylhydrazine compound and 5-ethylindoline-2,3-dione as the isatin compound, Compound 29 was prepared.

### Example 31

### Preparation of 5-ethyl-5′-(3-methoxyphenyl)-3′H-spiro[indole-3,2′-[1,3,4]thiadiazol]-2(1H)-one (Compound 30)

Using the method set forth in Example 1 and using 3-methoxybenzothiohydrazide as the thiobenzoylhydrazine compound and 5-ethylindoline-2,3-dione as the isatin compound, Compound 30 was prepared.

### Example 32

### Preparation of 5′-phenyl-5-(trifluoromethoxy)-3′H-spiro[indole-3,2′-[1,3,4]thiadiazol]-2(1H)-one (Compound 31)

Using the method set forth in Example 1 and using benzothiohydrazide as the thiobenzoylhydrazine compound and 5-(trifluoromethoxy)isopropylindoline-2,3-dione as the isatin compound, Compound 31 was prepared.

### Example 33

### Preparation of 5-methyl-1-(2-methylphenyl)-5′-(4-methylphenyl)-3′H-spiro[indole-3,2′-[1,3,4]thiadiazol]-2(1H)-one (Compound 32)

Using the method set forth in Example 1 and using 4-methylbenzothiohydrazide as the thiobenzoylhydrazine compound and 1-(2-methylphenyl)-5-methylindoline-2,3-dione as the isatin compound, Compound 32 was prepared.

### Example 34

### Preparation of 1-benzyl-5-methyl-5′-(4-methylphenyl)-3′H-spiro[indole-3,2′-[1,3,4]thiadiazol]-2(1H)-one (Compound 33)

Using the method set forth in Example 1 and using 4-methylbenzothiohydrazide as the thiobenzoylhydrazine compound and 1-benzyl-5-methylindoline-2,3-dione as the isatin compound, Compound 33 was prepared.

### Example 35

### Preparation of 5,7-dimethyl-5′-phenyl-3′H-spiro[indole-3,2′-[1,3,4]thiadiazol]-2(1H)-one (Compound 34)

Using the method set forth in Example 1 and using benzothiohydrazide as the thiobenzoylhydrazine compound and 5,7-dimethylindoline-2,3-dione as the isatin compound, Compound 34 was prepared.

### Example 36

### Preparation of 5-methoxy-5′-phenyl-3′H-spiro[indole-3,2′-[1,3,4]thiadiazol]-2(1H)-one (Compound 35)

Using the method set forth in Example 1 and using benzothiohydrazide as the thiobenzoylhydrazine compound and 5-methoxyindoline-2,3-dione as the isatin compound, Compound 35 was prepared.

### Example 37

### Preparation of 1-(4-chlorobenzoyl)-5′-(4-methoxyphenyl)-3′H-spiro[indole-3,2′-[1,3,4]thiadiazol]-2(1H)-one (Compound 36)

Using the method set forth in Example 1 and using 4-methoxybenzothiohydrazide as the thiobenzoylhydrazine compound and 1-(4-chlorobenzoyl)indoline-2,3-dione as the isatin compound, Compound 36 was prepared.

### Example 38

### Preparation of 5′-(4-methylphenyl)-6-propyl-3′H-spiro[indole-3,2′-[1,3,4]thiadiazol]-2(1H)-one (Compound 37)

Using the method set forth in Example 1 and using 4-methylbenzothiohydrazide as the thiobenzoylhydrazine compound and 6-propylindoline-2,3-dione as the isatin compound, Compound 37 was prepared.

### Example 39

### Preparation of 1,5′-diphenyl-3′H-spiro[indole-3,2′-[1,3,4]thiadiazol]-2(1H)-one (Compound 38)

Using the method set forth in Example 1 and using benzothiohydrazide as the thiobenzoylhydrazine compound and 1-phenylindoline-2,3-dione as the isatin compound, Compound 38 was prepared.

### Example 40

### Preparation of 5,6-difluoro-5′-phenyl-3′H-spiro[indole-3,2′-[1,3,4]thiadiazol]-2(1H)-one (Compound 39)

Using the method set forth in Example 1 and using benzothiohydrazide as the thiobenzoylhydrazine compound and 5,6-difluoro-indoline-2,3-dione as the isatin compound, Compound 39 was prepared.

### Example 41

### Preparation of 4,7-dichloro-5′-phenyl-3′H-spiro[indole-3,2′-[1,3,4]thiadiazol]-2(1H)-one (Compound 40)

Using the method set forth in Example 1 and using benzothiohydrazide as the thiobenzoylhydrazine compound and 4,7-dichloro-indoline-2,3-dione as the isatin compound, Compound 40 was prepared.

### Example 42

### Preparation of 1-allyl-5′-phenyl-3′H-spiro[indole-3,2′-[1,3,4]thiadiazol]-2(1H)-one (Compound 41)

Using the method set forth in Example 1 and using benzothiohydrazide as the thiobenzoylhydrazine compound and 1-allyl-indoline-2,3-dione as the isatin compound, Compound 41 was prepared.

### Example 43

### Preparation of 1-isopropyl-5′-phenyl-3′H-spiro[indole-3,2′-[1,3,4]thiadiazol]-2(1H)-one (Compound 42)

Using the method set forth in Example 1 and using benzothiohydrazide as the thiobenzoylhydrazine compound and 1-isopropyl-indoline-2,3-dione as the isatin compound, Compound 42 was prepared.

### Example 44

### Preparation of 5-ethyl-5′-(4-fluorophenyl)-3′H-spiro[indole-3,2′-[1,3,4]thiadiazol]-2(1H)-one (Compound 43)

Using the method set forth in Example 1 and using 4-fluoro-benzothiohydrazide as the thiobenzoylhydrazine compound and 5-ethyl-indoline-2,3-dione as the isatin compound, Compound 43 was prepared.

### Example 45

### Preparation of 5′-(4-tert-butylphenyl)-5-ethyl-3′H-spiro[indole-3,2′-[1,3,4]thiadiazol]-2(1H)-one (Compound 44)

Using the method set forth in Example 1 and using 4-tert-butyl-benzothiohydrazide as the thiobenzoylhydrazine compound and 5-ethyl-indoline-2,3-dione as the isatin compound, Compound 44 was prepared.

### Example 46

### Preparation of 1-butyryl-5-ethyl-5′-(4-methylphenyl)-3′H-spiro[indole-3,2′-[1,3,4]thiadiazol]-2(1H)-one (Compound 45)

Using the method set forth in Example 1 and using 4-methyl-benzothiohydrazide as the thiobenzoylhydrazine compound and 1-butyryl-5-ethyl-indoline-2,3-dione as the isatin compound, Compound 45 was prepared.

### Example 47

### Preparation of 1-butyryl-5-ethyl-5′-(4-methoxyphenyl)-3′H-spiro[indole-3,2′-[1,3,4]thiadiazol]-2(1H)-one (Compound 46)

Using the method set forth in Example 1 and using 4-methoxybenzothiohydrazide as the thiobenzoylhydrazine compound and 1-butyryl-5-ethyl-indoline-2,3-dione as the isatin compound, Compound 46 was prepared.

### Example 48

### Preparation of 5-ethyl-5′-phenyl-3′H-spiro[indoline-3,2′-[1,3,4]thiadiazol]-2-one (Compound 47)

Using the method set forth in Example 1 and using benzothiohydrazide as the thiobenzoylhydrazine compound and 5-ethyl-indoline-2,3-dione as the isatin compound, Compound 47 was prepared.

### Example 49

### Preparation of 5-ethyl-5′-(4-methylphenyl)-3′H-spiro[indoline-3,2′-[1,3,4]thiadiazol]-2-one (Compound 48)

Using the method set forth in Example 1 and using 4-methylbenzothiohydrazide as the thiobenzoylhydrazine compound and 5-ethyl-indoline-2,3-dione as the isatin compound, Compound 48 was prepared.

### Example 50

### Preparation of 5-methyl-5′-(4-methylphenyl)-3′H-spiro[indoline-3,2′-[1,3,4]thiadiazol]-2-one (Compound 49)

Using the method set forth in Example 1 and using 4-methylbenzothiohydrazide as the thiobenzoylhydrazine compound and 5-methyl-indoline-2,3-dione as the isatin compound, Compound 49 was prepared.

### Example 51

### Preparation of 5′-(4-methylphenyl)-3′H-spiro[indoline-3,2′-[1,3,4]thiadiazol]-2-one (Compound 50)

Using the method set forth in Example 1 and using 4-methylbenzothiohydrazide as the thiobenzoylhydrazine compound and indoline-2,3-dione as the isatin compound, Compound 50 was prepared.

### Example 52

### Preparation of 1-methyl-5′-(4-methylphenyl)-3′H-spiro[indoline-3,2′-[1,3,4]thiadiazol]-2-one (Compound 51)

Using the method set forth in Example 1 and using 4-methylbenzothiohydrazide as the thiobenzoylhydrazine compound and 1-methylindoline-2,3-dione as the isatin compound, Compound 51 was prepared.

### Example 53

### Preparation of 1-methyl-5′-phenyl-3′H-spiro[indoline-3,2′-[1,3,4]thiadiazol]-2-one (Compound 52)

Using the method set forth in Example 1 and using benzothiohydrazide as the thiobenzoylhydrazine compound and 1-methylindoline-2,3-dione as the isatin compound, Compound 52 was prepared.

### Example 54

### Preparation of 5′-phenyl-3′H-spiro[indoline-3,2′-[1,3,4]thiadiazol]-2-one (Compound 53)

Using the method set forth in Example 1 and using benzothiohydrazide as the thiobenzoylhydrazine compound and indoline-2,3-dione as the isatin compound, Compound 53 was prepared.

### Example 55

### Preparation of 5-bromo-5′-phenyl-3′H-spiro[indoline-3,2′-[1,3,4]thiadiazol]-2-one (Compound 54)

Using the method set forth in Example 1 and using benzothiohydrazide as the thiobenzoylhydrazine compound and 5-bromoindoline-2,3-dione as the isatin compound, Compound 54 was prepared.

### Example 56

### Preparation of 5-methyl-5′-phenyl-3′H-spiro[indoline-3,2′-[1,3,4]thiadiazol]-2-one (Compound 55)

Using the method set forth in Example 1 and using benzothiohydrazide as the thiobenzoylhydrazine compound and 5-methylindoline-2,3-dione as the isatin compound, Compound 55 was prepared.

### Example 57

### Preparation of 1-acetyl-5′-phenyl-3′H-spiro[indoline-3,2′-[1,3,4]thiadiazol]-2-one (Compound 56)

Using the method set forth in Example 1 and using benzothiohydrazide as the thiobenzoylhydrazine compound and 1-N-acetyl-indoline-2,3-dione as the isatin compound, Compound 56 was prepared.

### Example 58

### Mass Spectrometry Data for Illustrative Thiadiazole Compounds

Mass spectrometry data was obtained for illustrative Thiadiazole Compounds using an Agilent 1100 LC/MS instrument with an Agilent MSD detector employing the following MSD conditions (the corresponding HPLC conditions are given as (1.) in Example 59, below):

Ionization Mode: API-ES;

Gas Temperature: 350° C.;

Drying Gas: 11.0 L/min.;

Nebulizer Pressure: 55 psig;

Polarity: 50% positive, 50% negative;

VCap: 3000V (positive), 2500V (negative);

Fragmentor: 80 (positive), 120 or 140 (negative);

Mass Range: 100-1000 m/z;

Threshold: 150;

Step size: 0.15;

Gain: 1; and

Peak width: 0.15 min.

Mass spectrometry data for illustrative Thiadiazole Compounds listed in Table 1, below, whose listed HPLC retention times exceed 5 minutes, were obtained using alternative conditions on a Waters ZQ LC/MS instrument. The conditions were modified from those given above as follows (the corresponding alternative HPLC conditions are given as (2.) in Example 59, below): Mass spectrometry detector: Waters ZQ2000 MSD; Mass Range 100-700, Capillary 3.2 KV, Cone Voltage 20V, Multiplier 500.

### Example 59

### Calculation of HPLC Retention Times for Illustrative Thiadiazole Compounds

HPLC retention times were calculated for illustrative Thiadiazole Compounds whose retention times listed in Table 1, below, are less than 5 minutes, using an Agilent 1100 instrument and employing the following conditions (1.):

(1.) Column: Thermo Aquasil C18, 50×2.1 mm, 5 μm
Mobile Phase A: 0.1% Formic Acid in water
   B: 0.1% Formic Acid in ACN
Flow Rate: 0.800 mL/min
Column Temperature: 40° C.
Injection Volume: 5 mL
UV: monitor 215, 230, 254, 280, and 300 nm
Purity is reported at 254 nm unless otherwise noted
Solvent Gradient Table:

| Time (min) | % B (balance is % A) |
|---|---|
| 0 | 5 |
| 2.5 | 95 |
| 4.0 | 95 |
| 4.1 | 5 |
| 5.5 | 5 |

HPLC retention times were calculated for illustrative Thiadiazole Compounds whose retention times listed in Table 1, below, exceed 5 minutes, using alternative HPLC conditions (2.) and employing a Waters ZQ LC/MS instrument. The alternative HPLC conditions (2.) were as follows:

(2.) Column: Waters Xterra MS C18 50 mm×2.1 mm i.d., 3.5 μm; or Luna C18 30 mm×2.1 mm i.d., 2.5 μm
Flow Rate: 0.25 ml/min
Run Time: 11 min
Column Temperature Ambient
UV: monitor 254 nm
Eluent: A) 95/5 Water/Acetonitrile+0.1% TFA
   B) 5/95 Water/Acetonitrile+0.1% TFA
Solvent Gradient Table:

| Time (min) | % Solvent |
|---|---|
| 0 min | 100% Phase A, 0% Phase B |
| 1 min | 100% Phase A, 0% Phase B |
| 5 min | 0% Phase A, 100% Phase B |
| 9 min | 0% Phase A, 100% Phase B |
| 9.1 min | 100% Phase A, 0% Phase B |
| 11 min | 100% Phase A, 0% Phase B |

Table 1, below, sets forth mass spectrometry data and HPLC retention times for Illustrative Thiadiazole Compounds, using the methods set forth in Examples 47 and 48, respectively.

TABLE 1

| | | LC/MS |
|---|---|---|
| | Retention | MW |
| Compound | Time (min) | Observed |
| 1 | 2.41 | 340 |
| 2 | 2.30 | 326 |
| 3 | 2.18 | 312 |
| 4 | 2.37 | 390 |
| 5 | 2.29 | 326 |
| 6 | 2.18 | 342 |
| 7 | 2.87 | 350.1 |
| 8 | 2.36 | 398.1 |
| 9 | 2.51 | 402.1 |
| 10 | 2.57 | 383.1 |
| 11 | 2.29 | 344 |
| 12 | 2.27 | 344 |
| 13 | 2.74 | 486.2 |
| 14 | 2.74 | 486.2 |
| 15 | 2.41 | 354.1 |
| 16 | 2.18 | 355.1 |
| 17 | 2.38 | 358.1 |
| 18 | 2.24 | 326.1 |
| 19 | 2.24 | 326.1 |
| 20 | 2.25 | 356.1 |
| 21 | 2.86 | 456.5 |
| 22 | 2.59 | 416.2 |
| 23 | 2.60 | 416.1 |
| 24 | 2.62 | 416.1 |
| 25 | 2.64 | 436.1 |
| 26 | 2.70 | 470 |
| 27 | 2.58 | 447.1 |
| 28 | 2.68 | 444.2 |
| 29 | 6.52 | 340.1 |
| 30 | 7.54 | 340.1 |
| 31 | 6.45 | 366.1 |
| 32 | 2.65 | 400.1 |
| 33 | 3.59 | 400 |
| 34 | 2.84 | 309.9 |
| 35 | 2.51 | 311.9 |
| 36 | 2.64 | 456.1 |
| 37 | 2.46 | 338.1 |
| 38 | 2.50 | 358.1 |
| 39 | 2.24 | 316 |
| 40 | 7.38 | 350 |
| 41 | 6.47 | 322.1 |
| 42 | 6.51 | 324.2 |
| 43 | 7.28 | 328.1 |
| 44 | 8.42 | 366.2 |
| 45 | 8.92 | 394.2 |
| 46 | 8.57 | 410.2 |
| 47 | 2.35 | 310 |
| 48 | 2.53 | 324.1 |
| 49 | 2.36 | 310 |
| 50 | 2.6 | 294.4 |
| 51 | 2.28 | 310.1 |
| 52 | 2.66 | 295.5 |
| 53 | 2.13 | 282 |
| 54 | 2.83 | 361.7 |
| 55 | 2.67 | 295.5 |
| 56 | 7.42 | 324.1 |

### Example 60

### Aggrecanase 2 Inhibitory Effects of Illustrative Thiadiazole Compounds

A continuous vitro fluorescence assay was employed, wherein the enzyme used in the assay was a purified recombinant human Aggrecanase-2 (Agg2-Phe₆₂₈) (MW=41,737), which is a truncated form of full-length human aggrecanase-2 that was expressed in CHO cells and purified prior to use. The final concentration of Agg2-Phe₆₂₈ was 0.5 μg/ml. The buffer used in this assay was 50 mM HEPES, pH 7.5, 100 mM NaCl, 5 mM CaCl₂, 0.1% CHAPS, 5% glycerol. The substrate used in the assay was a synthetic peptide having the sequence ABz-TESESRGAIY-Dap(Dnp)-KK—NH₂ (>95% pure by HPLC, AnaSpec, Inc.), wherein Abz refers to ortho-aminobenzyl, a fluorescent group which is quenched by energy transfer to a 2,4-dinitrophenyl group. The final concentration of substrate in the assay was 25 μM and was spectrophotometrically determined using the extinction coefficient at 354 nm of 18,172 M⁻¹cm⁻¹. The Vₘₐₓ and Kₘ for this enzyme/substrate reaction were determined to be insensitive to DMSO up to at least 10% (v/v).

An illustrative Thiadiazole Compound (in duplicate) was serially diluted from 2 mM to 0.01 μM in DMSO. The total reaction volume was 100 μl. The buffer and enzyme were added first followed by addition of 10× inhibitor from the dilution plate. Separate enzyme and buffer samples were included in order to obtain the maximal rate of substrate cleavage. The reaction was allowed to stand at 30° C. for 15 minutes, then substrate was added, the resultant solution was mixed. The resultant reaction was allowed to progress, with monitoring, for 40 minutes at 30° C. in kinetic mode using λₑₓ: 340 nm and λₑₘ: 420 (GeminiXS Molecular Devices).

N2-{(2R)-2-[(1S)-1-Hydroxycarbamoyl-2-(thiophen-2-ylsulfanyl)-ethyl]-4-methyl-pentanoyl}-N-methyl-L-phenylalaninamide, a hydroxamic acid with an Agg2 IC₅₀ of 36+/−4 nM was run on each plate (in duplicate) as a positive control for the assay at the following concentrations: 200 nM, 20 nM and 10 nM which correspond to 100, 60 and 40% inhibition of Agg2-Phe₆₂₈ activity respectively.

### Data Analysis

The fluorescence intensity is linear during the time of data collection. The slope of the line (Vmax/sec) represents the initial reaction rate, ν. The maximal rate of cleavage of substrate was determined in the absence of inhibitor. The percent inhibition of activity in the presence of inhibitor was calculated using the following equation:

% inhibition=(1−ν(RFU/sec))/Maximal Rate(RFU/sec))*100

The IC₅₀ was obtained by fitting the initial rate, ν or % inhibition at each concentration of inhibitor to the following equation:

y=(a−d)/(1+C/IC₅₀)̂n)+d

This model describes a sigmoidal curve with an adjustable baseline, wherein:

y is the % inhibition or initial rate of reaction;

C is the concentration of inhibitor under test;

a is the limiting response as C approaches zero. As C increases without bound y tends toward its lower limit, d;

y is halfway between the lower and upper asymptotes when C=IC₅₀; and

n is the Hill coefficient. The sign of n is positive when the response increases with increasing dose and is negative when the response decreases with increasing dose (inhibition).

Assay results obtained using this method are shown in Table 2 below.

TABLE 2

| | IC₅₀ | % |
|---|---|---|
| Compound | (nM) | Inhibition |
| 1 | 3059 | NA |
| 2 | >7000 | NA |
| 3 | 13430 | NA |
| 4 | 2408 | NA |
| 5 | 2533 | NA |
| 6 | >2500 | NA |
| 7 | 2471 | NA |
| 8 | 1175 | NA |
| 9 | 3678 | NA |
| 10 | 874 | NA |
| 11 | NA | 30 |
| 12 | 1186 | NA |
| 13 | 1957 | NA |
| 14 | NA | 50 |
| 15 | 775 | NA |
| 16 | 4430 | NA |
| 17 | 1040 | NA |
| 18 | 3747 | NA |
| 19 | 1400 | NA |
| 20 | 5924 | NA |
| 21 | 5418 | NA |
| 22 | 644 | NA |
| 23 | 899 | NA |
| 24 | NA | 39 |
| 25 | 1483 | NA |
| 26 | 1257 | NA |
| 27 | 995 | NA |
| 28 | 950 | NA |
| 29 | 1361 | NA |
| 30 | 5930 | NA |
| 31 | 7220 | NA |
| 32 | 1221 | NA |
| 33 | 612 | NA |
| 34 | 4671 | NA |
| 35 | NA | 41 |
| 36 | NA | 48 |
| 37 | 1396 | NA |
| 38 | 2615 | NA |
| 39 | 14338 | NA |
| 40 | NA | 39 |
| 41 | 4876 | NA |
| 42 | 3051 | NA |
| 43 | 2965 | NA |
| 44 | 1660 | NA |
| 45 | 1447 | NA |
| 46 | 1523 | NA |
| 47 | NA | 9.6 |
| 48 | NA | 1.9 |
| 49 | NA | 3 |
| 50 | NA | 3.7 |
| 51 | NA | 4.6 |
| 52 | 24133 | NA |
| 53 | 30040 | NA |
| 54 | 13592 | NA |
| 55 | 11137 | NA |
| 56 | 12473 | NA |

NA = not available

### Example 61

### In Vitro Fluorescence Assay of MMP-13 Activity

### Materials and Methods

A continuous assay is used in which the substrate is a synthetic peptide containing a fluorescent group (7-methoxycoumarin; Mca), which is quenched by energy transfer to a 2,4-dinitrophenyl group. When the peptide is cleaved by MMP, a large increase in fluorescence is observed. The source of enzyme in the assay is the recombinant human catalytic domain of MMP-13 (165 amino acids, residues 104-268, 19 kDa) prepared at Wyeth-Research in Cambridge. The substrate used is the peptide Mca-PQGL-(3-[2,4-dinitrophenyl]-L-2,3-diaminopropionyl)-AR—OH. The assay buffer consists of 50 mM Hepes (pH 7.4), 100 mM NaCl, 5 mM CaCl₂, and 0.005% Brij-35. Each well of a 96-well plates contains a 200 μL reaction mixture consisting of assay buffer, purified MMP (final concentration of 0.5 nM, prepared by diluting with the assay buffer), and varied concentrations of inhibitor (prepared by serially diluting a given inhibitor in DMSO in 96-well polypropylene plate). The plates are then incubated at 30° C. for 15 minutes. The enzymatic reactions are initiated by adding the substrate to a final concentration of 20 μM, and mixing 10 times with a pipette. The final DMSO concentration in the assay is about 6.0%. The initial rate of the cleavage reaction can be determined at 30° C. using a fluorescence plate reader (excitation filter of 330 nm and emission filter of 395 nm) immediately after substrate addition.

### Results

Plots of the inhibitor concentration vs. the percent inhibition are fit to the following equation: y=(a−d)/[¹⁺(x/c)^{b}]+d, a general sigmoidal curve with Hill slope, a to d. x is the inhibitor concentration under test. y is the percent inhibition. a is the limiting response as x approaches zero. As x increases without bound, y tends toward its limit d. c is the inflection point (IC₅₀) for the curve. That is, y is halfway between the lower and upper asymptotes when, x=c. b is the slope factor or Hill coefficient. (See, Knight, et al., FEBS Lett., 296, 263-266, (1992)).

### Example 62

### In Vitro Fluorescence Assay of MMP-14 Activity

### Materials and Materials

A continuous assay is used in which the substrate is a synthetic peptide containing a fluorescent group (7-methoxycoumarin; Mca), which is quenched by energy transfer to a 2,4-dinitrophenyl group. When the peptide is cleaved by MMP, a large increase in fluorescence was observed. The source of enzyme in the assay is the recombinant human catalytic domain of MMP-14 (177 amino acids corresponding to Tyr89-Gly265 of mature human enzyme; 20 kDa; Chemicon International, Inc. (catalog number CC1041)). The substrate used is the peptide Mca-PQGL-(3-[2,4-d]nitrophenyl-L-2,3-diaminopropionyl)-AR—OH. The assay buffer consists of 50 mM Hepes (pH 7.4), 100 mM NaCl, 5 mM CaCl₂, and 0.005% Brij-35. Each well of the 96-well plates contains a 200 μL reaction mixture consisting of assay buffer, MMP (final concentration of 25 ng/ml, prepared by diluting with the assay buffer), and varied concentrations of inhibitor (prepared by serially diluting a given inhibitor in DMSO in 96-well polypropylene plate). The plates are then incubated at 30° C. for 15 minutes. The enzymatic reactions are initiated by adding the substrate to a final concentration of 20 μM, and mixing 10 times with a pipette. The final DMSO concentration in the assay is about 6.0%. The initial rate of the cleavage reaction is determined at 30° C. using a fluorescence plate reader (excitation filter of 330 nm and emission filter of 395 nm) immediately after substrate addition.

### Results

Plots of the inhibitor concentration vs. the percent inhibition are fit to the following equation: y=(a−d)/[¹⁺(x/c)^{b}]+d, a general sigmoidal curve with Hill slope, a to d. x is the inhibitor concentration under test. y is the percent inhibition. a is the limiting response as x approaches zero. As x increases without bound, y tends toward its limit d. c is the inflection point (IC₅₀) for the curve. That is, y is halfway between the lower and upper asymptotes when x=c. b is the slope factor or Hill coefficient. (See, Knight, et al., FEBS Lett., 296, 263-266, (1992)).

### Example 63

### In Vitro Fluorescence Assay of MMP-2 Activity

### Materials and Methods

A continuous assay is used in which the substrate is a synthetic peptide containing a fluorescent group (7-methoxycoumarin; Mca), which is quenched by energy transfer to a 2,4-dinitrophenyl group. When the peptide is cleaved by MMP, a large increase in fluorescence is observed. The source of enzyme in the assay is the recombinant human MMP-2 (66 kDa; Oncogene Research Products (catalog number PF023 from Calbiochem)). The substrate used is the peptide Mca-PQGL-(3-[2,4-dinitrophenyl]-L-2,3-diaminopropionyl)-AR—OH. The assay buffer consists of 50 mM Hepes (pH 7.4), 100 mM NaCl, 5 mM CaCl₂, and 0.005% Brij-35. Each well of a 96-well plate contains a 200 μL reaction mixture consisting of assay buffer, MMP (final concentration of 25 ng/ml, prepared by diluting with the assay buffer), and varied concentrations of inhibitor (prepared by serially diluting a given inhibitor in DMSO in 96-well polypropylene plate). The plates are then incubated at 30° C. for 15 minutes. The enzymatic reactions are initiated by adding the substrate to a final concentration of 20 μM, and mixing 10 times with a pipette. The final DMSO concentration in the assay is about 6.0%. The initial rate of the cleavage reaction is determined at 30° C. using a fluorescence plate reader (excitation filter of 330 nm and emission filter of 395 nm) immediately after substrate addition.

### Results

Plots of the inhibitor concentration vs. the percent inhibition are fit to the following equation: y=(a−d)/[¹⁺(x/c)^{b}]+d, a general sigmoidal curve with Hill slope, a to d. x is the inhibitor concentration under test. y is the percent inhibition. a is the limiting response as x approaches zero. As x increases without bound, y tends toward its limit d. c is the inflection point (IC₅₀) for the curve. That is, y is halfway between the lower and upper asymptotes when x=c. b is the slope factor or Hill coefficient. (See, Knight, C. G., Willenbrock, F., and Murphy, G. FEBS Lett. (1992) 296, 263-266).

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound having the Formula: 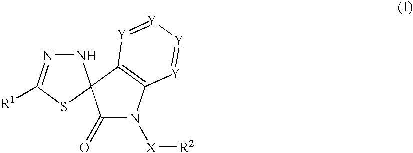
or a pharmaceutically acceptable salt or hydrate thereof,
wherein
X is —(CH₂)ₘ—C(O)O—, —(CH₂)ₘ—C(O)NH— or —(CH₂)ₘ—SO₂—;
m is 0 or 1;
each Y is —C(R³)—;
R¹ is -aryl or -5 or 6-membered aromatic or non-aromatic heterocycle, wherein the -aryl or -5 or 6-membered aromatic or non-aromatic heterocycle group is unsubstituted or substituted with one or more R⁴ groups;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

2. A compound having the Formula: 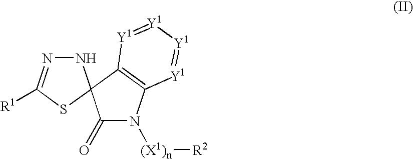
or a pharmaceutically acceptable salt or hydrate thereof,
wherein
X¹ is —CH₂—, —(CH₂)ₘ—C(O)—, —(CH₂)ₘ—C(O)NR²—, —(CH₂)ₘ—C(O)O—, —(CH₂)ₘ—C(O)NH— or —(CH₂)ₘ—SO₂—;
m is 0 or 1;
n is 0 or 1;
each Y¹ is independently —C(R³)— or —N—, wherein at least one occurrence of Y¹ is —N—;
R¹ is -aryl or -5 or 6-membered aromatic or non-aromatic heterocycle, wherein the -aryl or -5 or 6-membered aromatic or non-aromatic heterocycle group is unsubstituted or substituted with one or more R⁴ groups;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

3. A compound having the Formula: 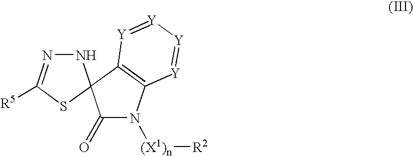
or a pharmaceutically acceptable salt or hydrate thereof,
wherein
X¹ is —CH₂—, —(CH₂)ₘ—C(O)—, —(CH₂)ₘ—C(O)NR²—, —(CH₂)ₘ—C(O)O—, —(CH₂)ₘ—C(O)NH— or —(CH₂)ₘ—SO₂—;
m is 0 or 1;
n is 0 or 1;
each Y is independently —C(R³)— or —N—;
R⁵ is -5 or 6-membered aromatic or non-aromatic heterocycle, which is unsubstituted or substituted with one or more R⁴ groups;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic, or non-aromatic heterocycle or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂;
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R⁴ is independently -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

4. A compound having the Formula: 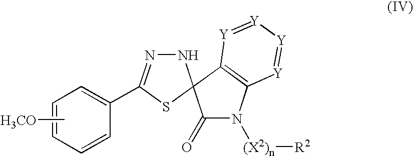
or a pharmaceutically acceptable salt or hydrate thereof,
wherein
X² is —CH₂—, —(CH₂)ₘ—C(O)O—, —(CH₂)ₘ—C(O)NH—, —(CH₂)ₘ—C(O)NR²—, or —(CH₂)ₘ—SO₂—;
each Y is independently —C(R³)— or —N—;
m is 0 or 1;
n is 0 or 1;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂,
wherein the —OCH₃ depicted in Formula (IV) occupies the para position or an ortho or meta position on the phenyl ring to which it is attached.

5. A compound having the Formula: 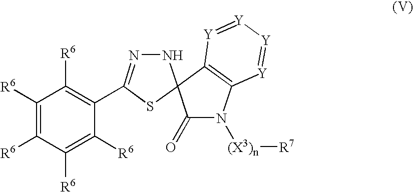
or a pharmaceutically acceptable salt or hydrate thereof,
wherein
X³ is —CH₂—, —(CH₂)ₘ—C(O)O—, —(CH₂)ₘ—C(O)NH—, or —(CH₂)ₘ—SO₂—;
each Y is independently —C(R⁸)— or —N—;
m is 0 or 1;
n is 0 or 1;
each R⁶ is independently —H, —C₁-C₆ alkyl, or -halo;
R⁷ is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein a —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), such that when n is 0, R⁷ is not —H; and
each R⁸ is independently —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂.

6. A compound having the Formula: 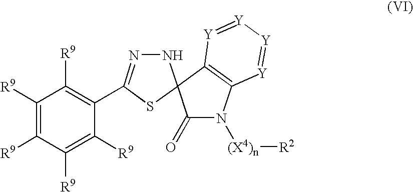
or a pharmaceutically acceptable salt or hydrate thereof,
wherein
X⁴ is —CH₂—, —C(O)—, —(CH₂)ₘ—C(O)O—, —(CH₂)ₘ—C(O)NH, or —(CH₂)ₘ—SO₂—;
each Y is independently —C(R³)— or —N—;
m is 0 or 1;
n is 0 or 1;
each R⁹ is independently —H, —C₁-C₆ alkyl, —O—C₁-C₆ alkyl, or -halo;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂,
such that:
every R⁹ and every R³ are not simultaneously —H when n is 0 and R² is —H, methyl, ethyl, butyl, pentyl, or an unsubstituted or mono- or di-substituted —(C₁-C₆ alkyl)-aryl;
R⁹ and R² are not simultaneously H when n is 0, exactly one R³ group is bromo, isopropyl, ethyl, or methyl and the other R³ groups are —H;
R² is not naphthyl, methyl, butyl, or pentyl when every R⁹ and R³ group is —H and n is 0;
R² is not —H or methyl when n is 0, exactly one R⁹ group is methyl while the other R⁹ groups are —H, and every R³ group is —H or exactly one R³ group is methyl or ethyl while the other R³ groups are —H;
R² is not naphthyl substituted with exactly one -halo or one —NO₂ group, or phenyl substituted with exactly one -halo or one —NO₂ group, when (X)ₙ is —CH₂— and every R⁹ and R³ group is —H or exactly one R⁹ group is methyl and the other R⁹ and R³ groups are —H;
R² is not —H, methyl, or —(C₁-C₆ alkyl)-(5 or 6-membered non-aromatic heterocycle) when exactly one R⁹ group is methyl, n is 0, and every R³ group is —H;
X⁴ is not —C(O)— and R² is not propyl or methyl when every R³ is —H, n is 1, and every R⁹ is —H or exactly one R⁹ is —O—(C₁-C₆ alkyl) and the other R⁹ groups are —H;
exactly one R³ group is not methyl, ethyl, or isopropyl when the other three R³ groups are —H, and each R⁹ group is —H or the R⁹ group at the para position is methyl and the other R⁹ groups are —H, and R² is —H; and
the R³ groups do not comprise exactly one methyl and exactly one -halo or the R³ groups do not comprise exactly two methyl groups at the 6 and 7 positions of the indoline ring when n is 0 and R² and every R⁹ are —H.

7. The compound of claim 4, wherein each occurrence of Y is —C(R³)—.

8. The compound of claim 4, wherein each occurrence of Y is —CH—.

9. The compound of claim 4, wherein the —OCH₃ depicted in Formula (IV) occupies the para position on the phenyl ring to which it is attached.

10. The compound of claim 4, wherein the —OCH₃ depicted in Formula (IV) occupies a meta position on the phenyl ring to which it is attached.

11. The compound of claim 4, wherein n is 0 and R² is —H.

12. The compound of claim 4, wherein n is 0 and R² is —C₁-C₆ alkyl, —C₂-C₆ alkynyl or —(C₁-C₆ alkyl)-aryl.

13. The compound of claim 8, wherein at least one occurrence of R³ is -halo, —C₁-C₆ alkyl, or —NO₂.

14. The compound of claim 5 wherein each occurrence of Y is —C(R⁸)—.

15. The compound of claim 6 wherein each occurrence of Y is —C(R³)—.

16. The compound of claim 15, wherein each occurrence of Y is —CH—.

17. The compound of claim 15, wherein each occurrence of R⁹ is —H.

18. The compound of claim 15, wherein n is 0 and R² is -aryl, —C₁-C₆ alkyl or —(C₁-C₆ alkyl)-aryl.

19. The compound of claim 15, wherein at least one occurrence of R³ is —C₁-C₆ alkyl, —CF₃, -5 or 6-membered aromatic or non-aromatic heterocycle or —OCF₃.

20. A compound being:
5′-(4-methoxyphenyl)-5,7-dimethyl-3′H-spiro[indole-3,2′-[1,3,4]thiadiazol]-2(1H)-one;
5′-(4-methoxyphenyl)-1-methyl-3′H-spiro[indole-3,2′-[1,3,4]thiadiazol]-2(1H)-one;
5′-(4-methoxyphenyl)-3′H-spiro[indole-3,2′-[1,3,4]thiadiazol]-2(1H)-one;
5-bromo-5′-(4-methoxyphenyl)-3′H-spiro[indole-3,2′-[1,3,4]thiadiazol]-2(1H)-one;
5′-(4-methoxyphenyl)-5-methyl-3′H-spiro[indole-3,2′-[1,3,4]thiadiazol]-2(1H)-one;
5-methoxy-5′-(4-methoxyphenyl)-3′H-spiro[indole-3,2′-[1,3,4]thiadiazol]-2(1H)-one;
5′-(4-methoxyphenyl)-1-prop-2-yn-1-yl-3′H-spiro[indole-3,2′-[1,3,4]thiadiazol]-2(1H)-one;
5′-(4-methoxyphenyl)-1-[3-(trifluoromethyl)phenyl]-3′H-spiro[indole-3,2′-[1,3,4]thiadiazol]-2(1H)-one;
ethyl[5′-(4-methoxyphenyl)-2-oxo-3′H-spiro[indole-3,2′-[1,3,4]thiadiazol]-1(2H)-yl]acetate;
1-benzyl-5′-(4-methoxyphenyl)-3′H-spiro[indole-3,2′-[1,3,4]thiadiazol]-2(1H)-one;
1-[2-(dimethylamino)ethyl]-5′-(4-methoxyphenyl)-3′H-spiro[indole-3,2′-[1,3,4]thiadiazol]-2(1H)-one;
6-chloro-5′-(4-methoxyphenyl)-3′H-spiro[indole-3,2′-[1,3,4]thiadiazol]-2(1H)-one;
5-chloro-5′-(4-methoxyphenyl)-3′H-spiro[indole-3,2′-[1,3,4]thiadiazol]-2(1H)-one;
4-(4-benzylpiperazin-1-yl)-5′-(4-methoxyphenyl)-3′H-spiro[indole-3,2′-[1,3,4]thiadiazol]-2(1H)-one;
5′-(4-methoxyphenyl)-6-propyl-3′H-spiro[indole-3,2′-[1,3,4]thiadiazol]-2(1H)-one;
5′-(4-methoxyphenyl)-5-nitro-3′H-spiro[indole-3,2′-[1,3,4]thiadiazol]-2(1H)-one;
6-chloro-5′-(4-methoxyphenyl)-7-methyl-3′H-spiro[indole-3,2′-[1,3,4]thiadiazol]-2(1H)-one;
5′-(4-methoxyphenyl)-7-methyl-3′H-spiro[indole-3,2′-[1,3,4]thiadiazol]-2(1H)-one;
5′-(4-methoxyphenyl)-6-methyl-3′H-spiro[indole-3,2′-[1,3,4]thiadiazol]-2(1H)-one;
7-methoxy-5′-(4-methoxyphenyl)-6-methyl-3′H-spiro[indole-3,2′-[1,3,4]thiadiazol]-2(1H)-one;
1-ethyl-5-fluoro-5′-(4-methoxyphenyl)-6-(4-methylpiperazin-1-yl)-3′H-spiro[indole-3,2′-[1,3,4]thiadiazol]-2(1H)-one;
1-benzyl-5′-(4-methoxyphenyl)-5-methyl-3′H-spiro[indole-3,2′-[1,3,4]thiadiazol]-2(1H)-one;
5′-(4-methoxyphenyl)-5-methyl-1-(2-methylphenyl)-3′H-spiro[indole-3,2′-[1,3,4]thiadiazol]-2(1H)-one;
5′-(4-methoxyphenyl)-5-methyl-1-(4-methylphenyl)-3′H-spiro[indole-3,2′-[1,3,4]thiadiazol]-2(1H)-one;
1-(4-chlorophenyl)-5′-(4-methoxyphenyl)-5-methyl-3′H-spiro[indole-3,2′-[1,3,4]thiadiazol]-2(1H)-one;
1-(3,4-dichlorophenyl)-5′-(4-methoxyphenyl)-5-methyl-3′H-spiro[indole-3,2′-[1,3,4]thiadiazol]-2(1H)-one;
5′-(4-methoxyphenyl)-5-methyl-1-(3-nitrophenyl)-3′H-spiro[indole-3,2′-[1,3,4]thiadiazol]-2(1H)-one;
1-benzyl-5′-(4-methoxyphenyl)-6-propyl-3′H-spiro[indole-3,2′-[1,3,4]thiadiazol]-2(1H)-one;
5-ethyl-5′-(4-methoxyphenyl)-3′H-spiro[indole-3,2′-[1,3,4]thiadiazol]-2(1H)-one;
5-ethyl-5′-(3-methoxyphenyl)-3′H-spiro[indole-3,2′-[1,3,4]thiadiazol]-2(1H)-one;
5′-phenyl-5-(trifluoromethoxy)-3′H-spiro[indole-3,2′-[1,3,4]thiadiazol]-2(1H)-one;
5-methyl-1-(2-methylphenyl)-5′-(4-methylphenyl)-3′H-spiro[indole-3,2′-[1,3,4]thiadiazol]-2(1H)-one;
1-benzyl-5-methyl-5′-(4-methylphenyl)-3′H-spiro[indole-3,2′-[1,3,4]thiadiazol]-2(1H)-one,
5,7-dimethyl-5′-phenyl-3′H-spiro[indole-3,2′-[1,3,4]thiadiazol]-2(1H)-one;
5-methoxy-5′-phenyl-3′H-spiro[indole-3,2′-[1,3,4]thiadiazol]-2(1H)-one;
1-(4-chlorobenzoyl)-5′-(4-methoxyphenyl)-3′H-spiro[indole-3,2′-[1,3,4]thiadiazol]-2(1H)-one;
5′-(4-methylphenyl)-6-propyl-3′H-spiro[indole-3,2′-[1,3,4]thiadiazol]-2(1H)-one;
1,5′-diphenyl-3′H-spiro[indole-3,2′-[1,3,4]thiadiazol]-2(1H)-one;
5,6-difluoro-5′-phenyl-3′H-spiro[indole-3,2′-[1,3,4]thiadiazol]-2(1H)-one;
4,7-dichloro-5′-phenyl-3′H-spiro[indole-3,2′-[1,3,4]thiadiazol]-2(1H)-one;
1-allyl-5′-phenyl-3′H-spiro[indole-3,2′-[1,3,4]thiadiazol]-2(1H)-one;
1-isopropyl-5′-phenyl-3′H-spiro[indole-3,2′-[1,3,4]thiadiazol]-2(1H)-one;
5-ethyl-5′-(4-fluorophenyl)-3′H-spiro[indole-3,2′-[1,3,4]thiadiazol]-2(1H)-one;
5′-(4-tert-butylphenyl)-5-ethyl-3′H-spiro[indole-3,2′-[1,3,4]thiadiazol]-2(1H)-one;
1-butyryl-5-ethyl-5′-(4-methylphenyl)-3′H-spiro[indole-3,2′-[1,3,4]thiadiazol]-2(1H)-one; or
1-butyryl-5-ethyl-5′-(4-methoxyphenyl)-3′H-spiro[indole-3,2′-[1,3,4]thiadiazol]-2(1H)-one;
or a pharmaceutically acceptable salt or hydrate thereof.

21. A composition comprising an effective dose of the compound or a pharmaceutically acceptable salt or hydrate of the compound of claim 1 and a physiologically acceptable vehicle.

22. A composition comprising an effective dose of the compound or a pharmaceutically acceptable salt or hydrate of the compound of claim 2 and a physiologically acceptable vehicle.

23. A composition comprising an effective dose of the compound or a pharmaceutically acceptable salt or hydrate of the compound of claim 3 and a physiologically acceptable vehicle.

24. A composition comprising an effective dose of the compound or a pharmaceutically acceptable salt or hydrate of the compound of claim 4 and a physiologically acceptable vehicle.

25. A composition comprising an effective dose of the compound or a pharmaceutically acceptable salt or hydrate of the compound of claim 5 and a physiologically acceptable vehicle.

26. A composition comprising an effective dose of the compound or a pharmaceutically acceptable salt or hydrate of the compound of claim 6 and a physiologically acceptable vehicle.

27. A composition comprising an effective dose of the compound or a pharmaceutically acceptable salt or hydrate of the compound of claim 20 and a physiologically acceptable vehicle.

28. A method of treating or preventing a disorder in a mammal in need thereof, which comprises administering an effective dose of the compound of claim 1 or a pharmaceutically acceptable salt or hydrate thereof, wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

29. A method of treating or preventing a disorder in a mammal in need thereof, which comprises administering an effective dose of the compound of claim 2 or a pharmaceutically acceptable salt or hydrate thereof, wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

30. A method of treating or preventing a disorder in a mammal in need thereof, which comprises administering an effective dose of the compound of claim 3 or a pharmaceutically acceptable salt or hydrate thereof, wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

31. A method of treating or preventing a disorder in a mammal in need thereof, which comprises administering an effective dose of the compound of claim 4, or a pharmaceutically acceptable salt or hydrate thereof wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

32. A method of treating or preventing a disorder in a mammal in need thereof, which comprises administering an effective dose of the compound of claim 5, or a pharmaceutically acceptable salt or hydrate thereof wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

33. A method of treating or preventing a disorder in a mammal in need thereof, which comprises administering an effective dose of the compound of claim 6, or a pharmaceutically acceptable salt or hydrate thereof wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

34. A method of treating or preventing a disorder in a mammal in need thereof, which comprises administering an effective dose of the compound of claim 20, or a pharmaceutically acceptable salt or hydrate thereof wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

35. A method of treating or preventing a disorder in a mammal in need thereof, which comprises administering an effective dose of a compound having the Formula: 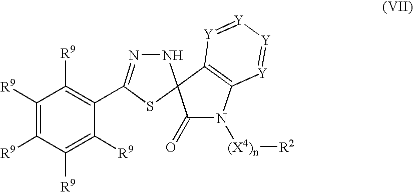
or a pharmaceutically acceptable salt or hydrate thereof,
wherein
X⁴ is —CH₂—, —C(O)—, —(CH₂)ₘ—C(O)O—, —(CH₂)ₘ—C(O)NH, —(CH₂)ₘ—C(O)NR², or —(CH₂)ₘ—SO₂—;
each Y is independently —C(R³)— or —N—;
m is 0 or 1;
n is 0 or 1;
each R⁹ is independently —H, —C₁-C₆ alkyl, —O—C₁-C₆ alkyl, or halo;
R² is —H, —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle), wherein the —C₁-C₆ alkyl, —C₂-C₆ alkenyl, —C₂-C₆ alkynyl, -aryl, —(C₁-C₆ alkyl)-aryl, -5 or 6-membered aromatic or non-aromatic heterocycle, or —(C₁-C₆ alkyl)-(5 or 6-membered aromatic or non-aromatic heterocycle) group is unsubstituted or substituted with one or more of the following groups: -halo, —O—C₁-C₆ alkyl, —O—C₂-C₆ alkenyl, —O—C₂-C₆ alkynyl, —O-aryl, —O—(C₁-C₆ alkyl)-aryl, —O-(5 or 6-membered aromatic or non-aromatic heterocycle), —O—(C₁-C₆ alkyl)-(5 or 6-membered aromatic or non-aromatic heterocycle), —CN, —C₁-C₆ alkyl, —C₂-C₆ alkenyl, —C₂-C₆ alkynyl, —CF₃, —OCF₃, —NO₂, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂; and
each R³ is independently —H, -halo, —OR², —CN, —C₁-C₆ alkyl, —C₂-C₆ alkenyl, —C₂-C₆ alkynyl, —CF₃, —OCF₃, —NO₂, —(C₁-C₆ alkyl)-(5 or 6-membered aromatic or non-aromatic heterocycle), -(5 or 6-membered aromatic or non-aromatic heterocycle)-aryl, -(5 or 6-membered aromatic or non-aromatic heterocycle)-CH₂-aryl, -(5 or 6-membered aromatic or non-aromatic heterocycle)-C₁-C₆ alkyl, —NH₂, —NH(C₁-C₆ alkyl), —N(C₁-C₆ alkyl)₂, —NHC(O)—(C₁-C₆ alkyl), —NHC(O)NH(C₁-C₆ alkyl), —SO₂NH₂, —SO₂NH(C₁-C₆ alkyl), —SO₂N(C₁-C₆ alkyl)₂, —NHSO₂(C₁-C₆ alkyl), —C(O)O—(C₁-C₆ alkyl), —C(O)NH(C₁-C₆ alkyl), or —C(O)N(C₁-C₆ alkyl)₂,
wherein the disorder is an arthritic disorder, osteoarthritis, cancer, rheumatoid arthritis, asthma, chronic obstructive pulmonary disease, atherosclerosis, age-related macular degeneration, myocardial infarction, a corneal ulceration, an ocular surface disease, hepatitis, an aortic aneurysm, tendonitis, a central nervous system disorder, abnormal wound healing, angiogenesis, restenosis, cirrhosis, multiple sclerosis, glomerulonephritis, graft versus host disease, diabetes, an inflammatory bowel disease, shock, invertebral disc degeneration, stroke, osteopenia, or a periodontal disease.

36. The method of claim 35, wherein the compound is:
5-ethyl-5′-phenyl-3′H-spiro[indole-3,2′-[1,3,4]thiadiazol]-2(1H)-one;
5-ethyl-5′-(4-methylphenyl)-3′H-spiro[indole-3,2′-[1,3,4]thiadiazol]-2(1H)-one;
5-methyl-5′-(4-methylphenyl)-3′H-spiro[indole-3,2′-[1,3,4]thiadiazol]-2(1H)-one;
5′-(4-methylphenyl)-3′H-spiro[indole-3,2′-[1,3,4]thiadiazol]-2(1H)-one;
1-methyl-5′-(4-methylphenyl)-3′H-spiro[indole-3,2′-[1,3,4]thiadiazol]-2(1H)-one;
1-methyl-5′-phenyl-3′H-spiro[indoline-3,2′-[1,3,4]thiadiazol]-2-one;
5′-phenyl-3′H-spiro[indoline-3,2′-[1,3,4]thiadiazol]-2-one;
5-bromo-5′-phenyl-3′H-spiro[indoline-3,2′-[1,3,4]thiadiazol]-2-one;
5-methyl-5′-phenyl-3′H-spiro[indoline-3,2′-[1,3,4]thiadiazol]-2-one; or
1-acetyl-5′-phenyl-3′H-spiro[indoline-3,2′-[1,3,4]thiadiazol]-2-one;
or a pharmaceutically acceptable salt or hydrate thereof.

37. The method of any one of claims 28-36, wherein the disorder is osteoarthritis.
